(19) United States
(12) Patent Application Publication
(10) Pub. No.: US 2014/0147887 A1
(43) Pub. Date: May 29, 2014

(54) NOVEL 12 ALPHA-HYDROXYSTEROID DEHYDROGENASES, PRODUCTION AND USE THEREOF

(71) Applicant: PharmaZell GmbH, Raubling (DE)

(72) Inventors: Arno Aigner, Tuntenhausen (DE); Ralf Gross, Rosenheim (DE); Rolf Schmid, Stuttgart (DE); Michael Braun, Bad Mergentheim-Edelfingen (DE); Steffen Mauer, Dirmstein (DE)

(73) Assignee: PharmaZell GmbH, Raubling (DE)

(21) Appl. No.: 13/969,069

(22) Filed: Aug. 16, 2013

Related U.S. Application Data
(63) Continuation of application No. 12/934,259, filed on Dec. 13, 2010, now abandoned.

(30) Foreign Application Priority Data
Mar. 26, 2006 (EP) 08153330.9

(51) Int. Cl. C12P 33/02

(52) U.S. Cl. CPC C12P 33/02
USPC 435/61

(57) ABSTRACT

The invention provides 12α-hydroxysteroid dehydrogenases, nucleic acid sequences coding for the same, expression cassettes and vectors, recombinant microorganisms containing the corresponding coding nucleic acid sequences, methods for producing said 12α-hydroxysteroid dehydrogenases, methods for enzymatic oxidation of 12α-hydroxysteroids using said enzyme, methods for enzymatic reduction of 12-ketosteroids using said enzyme, methods for qualitative or quantitative determination of 12-ketosteroids and/or 12α-hydroxysteroids using said 12α-hydroxysteroid dehydrogenases and methods for production of ursodesoxycholic acid, comprising the enzyme-catalysed cholic acid oxidation using said 12α-hydroxysteroid dehydrogenases.

### RELATED APPLICATIONS DATA

This application is a continuation of U.S. patent application Ser. No. 12/934,259, filed Dec. 13, 2010, now pending, which is the U.S. national phase application, pursuant to 35 U.S.C. §371, of PCT international application Ser. No. PCT/EP2009/002190, filed Mar. 25, 2009, designating the United States and published in German on Oct. 1, 2009 as publication WO 2009/118176 A2, which claims priority to European application Serial No. 08153330.9, filed Mar. 26, 2008. The entire contents of the aforementioned patent applications are incorporated herein by this reference.

The present invention relates to novel 12α-hydroxy-steroid dehydrogenases, nucleic acid sequences, expression cassettes and vectors coding therefor; recombinant microorganisms comprising appropriate encoding nucleic acid sequences; processes for the production of such 12α-hydroxysteroid dehydrogenases; processes for the enzymatic oxidation of 12α-hydroxysteroids using such enzymes, processes for the enzymatic reduction of 12-ketosteroids using such enzymes, processes for the qualitative or quantitative determination of 12-ketosteroids or 12α-hydroxysteroids using the 12α-hydroxysteroid dehydrogenases according to the invention; and a process for the preparation of ursodeoxycholic acid, comprising enzyme-catalyzed cholic acid oxidation using the 12α-hydroxysteroid dehydrogenases according to the invention.

### BACKGROUND OF THE INVENTION

12α-Hydroxysteroid dehydrogenase (12α-HSDH) (E.C. 1.1.1.176) is a biocatalyst important for stereospecific synthesis, such as, for example, the oxidation of cholic acid.

Investigations on a 12α-HSDH from Clostridium sp. group P strain 48-50 and its partial purification by NAD⁺ and NADP⁺ Sepharose chromatography was described by Mahony et al. (Mahony, D. E., et al. Appl Environ Microbiol, 1977, 34(4): p. 419-23) and MacDonald et al. (MacDonald, I. A., et al. Journal of Lipid Research, 1979, 20234-239).

A correspondingly prepared 12α-HSDH-containing protein extract from Clostridium group P was employed by Sutherland et al. in the course of three different synthesis routes of ursodeoxycholic acid from cholic acid. One of the synthesis routes here comprised the enzyme-catalyzed oxidation of cholic acid to 12-keto-chenodeoxycholic acid (12-keto-CDCA) (Sutherland, J. D., et al., Prep Biochem, 1982, 12(4): p. 307-21). Cell lysate from Clostridium sp. group P strain 48-50 DSM 4029 was used here as enzyme preparation. The reaction needs stoichiometric amounts of the cofactor NADP⁺.

The need for cofactor can be reduced by coupling with a cofactor-regenerating enzyme. For this, the prior art teaches, for example, the use of glutamate dehydrogenase (GLDH), which is co-immobilized together with a 12α-HSDH-containing protein extract Clostridium group P (Carrea, G., et al., Biotechnology and Bioengineering, 1984, 26(5): p. 560-563). The GLDH reoxidizes NADPH to NADP⁺ with simultaneous reductive amination of α-ketoglutarate to glutamate. Alternatively to this, alcohol dehydrogenases ADH-Tb, ADH-Lb and ADH-ms are also proposed for cofactor regeneration (Fossati, E., et al., Biotechnol Bioeng, 2006, 93(6): p. 1216-20). The ADH converts acetone to 2-propanol with regeneration of NADP⁺. For reaction on the 10 ml scale, not pure protein, but a 12α-HSDH-containing protein fraction with a specific activity of 12 U/mg of protein, enriched further from a commercial preparation (from ASA Spezialenzyme, Wolfenbüttel, Germany), was in turn employed there.

It is common to all investigations described above that the source for 12α-HSDH is the pathogenic and anaerobic strain Clostridium sp. group P strain 48-50. On account of the small proportion of this enzyme in the total protein (at most 1% of the total protein of Clostridium sp.), on the one hand access to industrially utilizable amounts of the enzyme is made difficult. On the other hand, its industrial production turns out to be complicated and costly, as the entire culturing and storage of the pathogenic production strain must be carried out in a plant that has a license for microbiological operations of risk group 2 (see BioStoffV).

Additionally, the pathogenicity of this production strain makes the use of 12α-HSDH in the synthesis of a pharmaceutical intermediate problematical. For the licensing of the production process according to GMP regulatory requirements a nonpathogenic enzyme source is necessary.

Cholic acid oxidation catalyzed by an NAD⁺-dependent 12α-HSDH, with cofactor regeneration by lactate dehydrogenase (LDH; conversion of pyruvate to lactate with regeneration of NAD⁺), is described in EP-A-1 731 618.

A two-stage purification strategy, based on a dye column affinity chromatography for the wild-type enzyme from Clostridium sp. group P strain 48-50 and an N-terminal amino acid sequence has furthermore been proposed (Braun, M., et al., Eur J Biochem, 1991, 196(2): p. 439-50). The N-terminal sequence published was a partial sequence comprising 29 amino acid residues, whose N-terminus reads as follows: Met-Ile-Phe-Asp-Gly-Lys-Val . . . . Moreover, no commercially obtainable column materials were used for the purification by this study group.

At present, a 12α-HSDH-containing extract from Clostridium sp. group P strain 48-50 is marketed by ASA Spezialenzyme, Wolfenbüttel, Germany. Investigations show, however, that the low specific activity of this commercial preparation complicates the extraction and work up of reaction products of the 12α-HSDH-catalyzed enzymatic reactions because of the high amount of total protein to be employed.

It is therefore the object of the present invention to make available a 12α-HSDH (in particular an NADP⁺-dependent enzyme) in a form that is suitable for preparative use in pharmaceutical active ingredient synthesis on the industrial scale, as, for example, in the enzyme-catalyzed oxidation of cholic acid to 12-ketochenodeoxycholic acid (12-keto-CDCA).

### DESCRIPTION OF THE FIGURES

In the attached figures,

FIG. 1 shows the gene and the protein sequence of the long version of 12α-HSDH(HSDH_long) according to the invention.

FIG. 2 shows the gene and the protein sequence of the short version of 12α-HSDH(HSDH_short) according to the invention and in comparison thereto the published incomplete partial sequence according to Braun, M., et al., loc. cit.

FIG. 3 shows the section of a multi-sequence alignment of known microbial HSDH and the HSDH according to the invention. The highly conserved positions are marked by “*”, the variable positions by “:”. Microbial hydroxysteroid dehydrogenases (the left column shows the associated Accession number and the organism of origin), whose function was investigated at the protein level or that have orthology in closely related species, were compared with the sequence HSDH_short (Csp2594) determined according to the invention. The known HSDH strains originate from Escherichia coli (ECOLI), Burkholderia mallei (BURM), Bacteroides fragilis (BACFR), Clostridium sordellii (CLOSO), Clostridium difficile (CLOD), Eubacterium sp. (EUBSP), Mycobacterium tuberculosis (MYCTU) and Streptomyces exfoliatus (STREX).

FIG. 4 shows the expression of 12α-HSDH enzymes according to the invention in cell lysates of BL21 and Rosetta™ (DE3) cells after 4 or 22 h. 12α-HSDH (“long” and “short”) were expressed for either 4 or 22 h in BL21 and Rosetta™ (DE3) cells. Subsequently, the cells were disrupted and 20 μg of protein were applied. The target protein (12α-HSDH) is to be found at 27 kDa.

FIG. 5 shows the course of the absorption at 340 nm during the preparation of 12K-CDCA on the 500 ml scale.

FIG. 6 shows a thin-layer chromatogram of reaction batches for the confirmation of the regioselectivity of the 12α-HSDH, where cholic acid (1) and 12-keto-chenodeoxycholic acid (2) were used as a reference; the reaction batches were thus compared with HSDH_long (3) and HSDH_short (4).

FIG. 7 shows the protein and nucleic acid sequence of the 12α-HSDH mutant 37D12.

FIG. 8 shows the homology model of the 12α-HSDH from Clostridium sp. DSM 4029 with bound NADPH, bound substrate and mutation position 97 (Gln97).

FIG. 9 shows a comparison of the activity of the 12α-HSDH and of the mutant 37D1. The activity of the 12α-HSDH and the mutant 37D12 at various percentage ratios (conversion in %) of cholic acid and 12-ketocheno-deoxycholic acid (total concentration 5 mM) is shown. The reaction was started with 0.25 mM NADP⁺. The change in the absorption was determined over 60 seconds, the activity being calculated over the range of 30 seconds which had the highest linearity (wild-type 0-30 seconds, 37D12 30-60 seconds). The relative HSDH activity refers to the activity with 5 mM cholic acid (0% conversion), whose absolute activity was set at 100%. The mean values and the standard errors of the activity measurement with N=3 are shown.

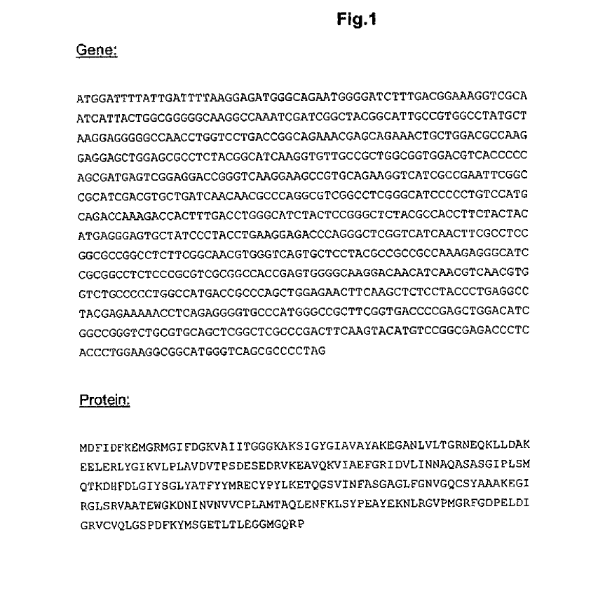

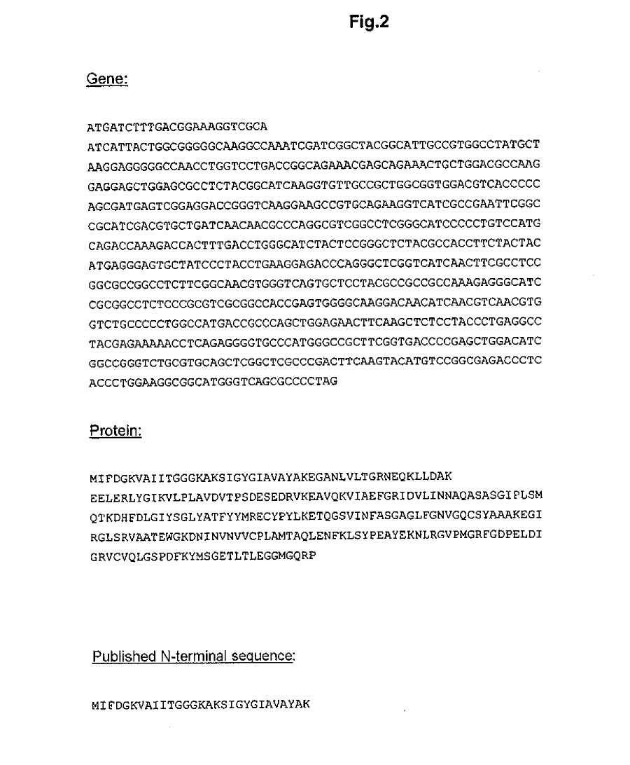

### SUMMARY OF THE INVENTION

The above object was surprisingly achieved by the elucidation for the first time of the encoding nucleic acid sequence and a description of the correct and complete amino acid sequence of the 12α-HSDH enzyme occurring in Clostridium group P, strain C48-50.

Surprisingly, it was found in particular that the N-terminal amino acid sequence described in the literature does not correspond to the actual N-terminal amino acid sequence, and that additionally the 12α-HSDH exists in a long version (HSDH_long) and in an N-terminally truncated short version (HSDH_short).

The achievement according to the invention of the above object appears all the more surprising as in the prior art it had not been recognized that 12α-HSDH enzymes of different length, in particular with a different N terminus, exist and the erroneous sequence information from the prior art prevented a correct primer synthesis and thus location and amplification of the encoding sequence.

Moreover, in addition to the studies of Braun, M. et al. loc. cit., more systematic investigations with published sequences for enzymes that belong to the class of short-chain dehydrogenases/reductases were carried out, to which, inter alia, 12α-HSDH also belongs. For this group of enzymes, a characteristic N-terminal sequence motif, namely T-G-X₃-G-X-G, was postulated by Oppermann et al. in Chemo-Biological Interactions, 2003, 143-144, 247-253. This sequence motif is not to be found in the published 12α-HSDH sequence from the year 1991 (Braun, M. et al. loc. cit.), because of which the reliability of the partial sequence information originally disclosed for 12α-HSDH has been questioned to date in the eye of the person skilled in the art.

### DETAILED DESCRIPTION OF THE INVENTION

### 1. Preferred Embodiments

A primary subject of the invention relates to pure, in particular recombinantly produced, 12α-hydroxysteroid dehydrogenases (12α-HSDHs) obtainable from Clostridium sp. with a molecular weight, determined by SDS-polyacrylamide gel electrophoresis (SDS-PAGE) under reducing conditions, in the range from more than about kD, in particular more than about 26.5, such as about 27 to 30, kD, and a calculated molecular weight of more than about 29 kD, in particular about 29.1 to 29.5 kD, such as, in particular, 29.359 kD for HSDH_long or approximately 27.8 for HSDH_short. The molecular weight details relate here to the molecular weight of the protein subunits of the enzyme; without being restricted thereto, the native protein consists, for example, of 4, in particular approximately equal-size, subunits of this type.

In particular, such a protein is obtainable from Clostridium sp. group P strain 48-50 (DSM4029). The enzyme can be prepared, for example, in a specific activity in the range of more than approximately 10, 15, 20 or 25 U/mg, such as, for example, 20 to 100 U/mg or 25 to 80 U/mg. The determination of the specific activity takes place here under the standard conditions specified in the experimental section.

The invention relates in particular to pure, in particular recombinantly produced, 12α-HSDHs of this type, comprising at least one of the following amino acid sequence motifs:

a)

(SEQ ID NO: 5)
LINN
(SEQ ID NO: 11)
RMGIFD

b)
c) N-terminal sequence, selected from

(SEQ ID NO: 6)
(1) MDFIDFKEMGRMGIFDGKVAIITGGGKAKSIGYGIAVAYAK
(SEQ ID NO: 7)
(2) MDFIDFKEMGRMGI
(SEQ ID NO: 8)
(3) ITGGGKAKSIGYGIA
(SEQ ID NO: 9)
(4) IFDGK
(SEQ ID NO: 10)
(5) GIFDGK

d) FGDPELDI (SEQ ID NO: 13) or sequences derived therefrom, such as, for example: GDPELDI, FGDPELD, DPELDI, FGDPEL, GDPEL, DPELD, GDPELD

Furthermore, the enzymes according to the invention are characterized in that they have no N-terminal (i.e. in the range of the N-terminal end of approximately 1 to amino acid residues) sequence motif of the type TGX₃GXG, in which X represents any desired amino acid residues.

The invention in particular relates to pure, in particular recombinantly produced, 12α-HSDHs,
a) comprising one of the amino acid sequences according to SEQ ID NO: 2 or 4, in each case beginning at position +1 or +2; or
b) comprising an amino acid sequence derived from a sequence according to a) with a percentage sequence identity of at least 60%; or
c) encoded by a nucleic acid sequence encoding a protein according to a) and b); or
d) encoded by an encoding nucleic acid sequence according to SEQ ID NO: 1 or 3; or by a sequence derived therefrom adapted to the respective codon utilization of an organism used for expression; or
e) encoded by an encoding sequence derived the nucleic acid sequences according to SEQ ID NO: 1 or 3, with a percentage sequence identity of at least 60%.

The adaptation of the nucleic acid sequence to the codon utilization can take place here according to customary methods, as accessible from, for example, from: http://slam.bs.jhmi.edu/cgi-bin/gd/gdRevTrans.cgi

The invention further relates to 12α-HSDH mutants with modified co-substrate utilization and/or reduced product inhibition; and in particular those mutants derived from a 12α-hydroxysteroid dehydrogenase according to the above definition, with at least one mutation modifying the co-substrate utilization in the sequence motif VLTGRNE (SEQ ID NO: 12). Nonlimiting examples of such mutants comprise those with at least one of the following amino acid substitutions in SEQ ID NO: 12: G→D; R→A; and mutants with at least one mutation reducing the product inhibition in the region of the amino acid residues forming the substrate binding pocket of the enzyme; such as, for example, comprising at least the mutation of amino acid Q, corresponding to position 97 and/or 99 of SEQ ID NO: 4 (corresponding to position 98 or 100 of SEQ ID NO: 22); in particular comprising a mutation corresponding to Q97H in SEQ ID NO: 4 (corresponding to Q98H in SEQ ID NO: 22).

Further potential amino acid substituents in position 98 (relative to SEQ ID NO: 22) comprise: A, N, D, C, E, G, H, M, S, T, V. Based on the homology model of the HSDH according to the invention, it is assumed that a substitution here leads to a weakening of the carboxyl binding of the product. Therefore the adjacent position S100 (based on SEQ ID NO: 22) was also mutated to the following amino acids: A, N, D, C, Q, E, G, H, M, T, V, K.

A group of mutants according to the invention thus comprises one or two mutations in position 97 or 99 (according to SEQ ID NO: 4) or in position 98 or 100 (according to SEQ ID NO: 22) selected from:

### Q→A, N, D, C, E, G, H, M, S, T, V;

### S→A, N, D, C, Q, E, G, H, M, T, V, K.

The invention relates in particular to 12α-HSDHs according to the above definition, obtainable by heterologous expression of at least one of the 12α-HSDH-encoding nucleic acid sequences described above, in particular those recombinantly produced enzymes, expressed in a nonpathogenic microorganism, such as, for example, expressed in a bacterium of the genus Escherichia, in particular of the species E. coli.

The invention moreover relates to nucleic acid sequences according to the above definition; expression cassettes, comprising at least one such encoding nucleic acid sequence under the genetic control of at least one regulative nucleic acid sequence; vectors, comprising at least one such expression cassettes; and according to recombinant microorganisms which carry at least one such nucleic acid sequence or expression cassette or is transformed with at least one such vector.

The invention additionally relates to a process for the production of a 12α-HSDH according to the above definition, where a recombinant microorganism according to the invention is cultured and the expressed 12α-HSDH is isolated from the culture.

The invention further relates to a process for the enzymatic oxidation of 12α-hydroxysteroids, where the hydroxysteroid is reacted in the presence of a 12α-HSDH according to the invention, and at least one oxidation product formed is optionally isolated from the reaction batch. The reaction can be carried out aerobically here (i.e. in the presence of oxygen) or anaerobically (i.e. essentially with exclusion of oxygen), in particular aerobically.

In particular, the hydroxysteroid here can is cholic acid (CA) or a cholic acid derivative, such as, in particular, a salt, amide or alkyl ester. Preferably, CA or a derivative thereof is reacted here to give 12-ketochenodeoxycholic acid (12-keto-CDCA) or to give the corresponding derivative. In particular, the reaction takes place takes place here in the presence and with stoichiometric consumption of NADP⁺ or NAD⁺.

The invention further relates to a process for the enzymatic reduction of 12-ketosteroids, where the ketosteroid is reacted in the presence of a 12α-HSDH according to the invention and a reduction product formed is optionally isolated from the reaction batch. The reaction can be carried out here aerobically or anaerobically, in particular aerobically.

The ketosteroid here is in particular 12-keto-CDCA or a derivative thereof, such as, in particular, a salt, amide or alkyl ester. The ketosteroid or its derivative here is reduced to the corresponding 12α-hydroxysteroid or its derivative. In particular, the reaction takes place here in the presence of NADPH or NADH.

In a preferred embodiment of the above redox reactions, the redox equivalents consumed can be regenerated electrochemically or enzymatically. Suitable enzymatic regeneration systems have already been described at the beginning. We expressly make reference to the disclosure of these publications. Nonlimiting examples thereof of suitable enzymes are glutamate dehydrogenase, alcohol dehydrogenase and lactate dehydrogenase. Electrochemical regeneration processes are based, for example, on hydridorhodium redox catalysts, as described, for example, in WO-A-01/88172, to which reference is hereby made.

In a further embodiment of the above redox reactions, these can take place with a 12α-HSDH in immobilized form. Enzymes optionally used for cofactor regeneration can likewise be immobilized.

The invention further relates to such a bioreactor for carrying out the above redox reactions, or those partial reaction steps in the course of a synthetic overall process.

The invention further relates to a process for the qualitative or quantitative detection of 12-ketosteroids or 12α-hydroxysteroids, where the steroid of a redox reaction catalyzed by a 12α-HSDH according to the invention is carried out in the presence of redox equivalents, a change in the concentration of the redox equivalents is determined and therefrom the content of 12-ketosteroids or 12α-hydroxysteroids is determined qualitatively or quantitatively.

The invention furthermore relates to processes for the synthesis of ursodeoxycholic acid (UDCA) from cholic acid (CA), comprising at least one reaction step catalyzed by a 12α-HSDH according to the invention. This reaction step can be carried out here aerobically or anaerobically, in particular aerobically. Three suitable reaction sequences have been described, for example, by Sutherland et al., loc. cit., to which reference is hereby made. The following synthesis routes 1 to 3 (where UCA represents ursocholic acid and CDCA represents chenodeoxycholic acid) have been described:

### 1st Route

| | |
|---|---|
| CA→12-keto-CDCA | (enzymatically by 12α-HSDH) |
| 12-keto-CDCA→12-keto-UDCA | (enzymatically by 7α- and 7β-HSDH) |
| 12-keto-UDCA→UDCA | (chemically, Wolff-Kishner reduction) |

### 2nd Route

| | |
|---|---|
| CA→UCA | (enzymatically by 7α- and 7β-HSDH) |
| UCA→12-keto-UDCA | (enzymatically by 12α-HSDH) |
| 12-keto-UDCA→UDCA | (chemically, Wolff-Kishner reduction) |

### 3rd Route

| | |
|---|---|
| CA→12-keto-CDCA | (enzymatically by 12α-HSDH) |
| 12-keto-CDCA→12-CDCA | (chemically, Wolff-Kishner reduction) |
| CDCA→UDCA | (whole Clostridium absonum cells) |

The invention relates in particular, however, to the following, 4th route:

### 4th Route

This relates to the preparation of an ursodeoxycholic acid of the formula (1)

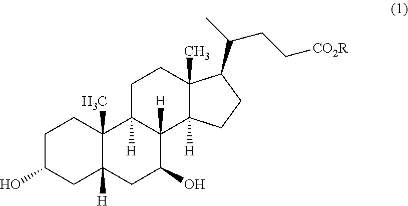

in which
R represents alkyl, NR¹R², H, an alkali metal ion or N(R³)₄⁺, in which the radicals R³ are identical or different and represent H or alkyl,
where
a) a cholic acid (CA) of the formula (2)

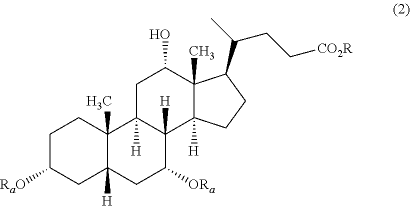

in which R has the meanings indicated above, and the radicals Rₐ are identical or different and represent H or acyl, is oxidized in the presence of a 12α-HSDH according to the invention to the corresponding 12-ketochenodeoxycholic acid (12-keto-CDCA) of the formula (3)

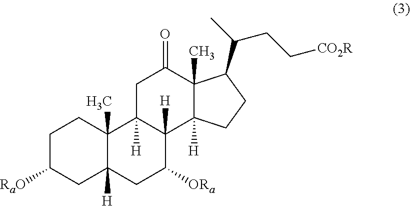

in which R and Rₐ have the meanings indicated above, and subsequently
b) 12-keto-CDCA of the formula (3) is reacted by deoxygenation, such as, for example, by Wolff-Kishner reduction, to give chenodeoxycholic acid (CDCA) of the formula (4)

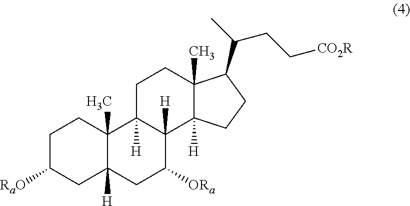

in which R and Rₐ have the meanings indicated above, and
c) CDCA of the formula (4) is chemically oxidized in position 7 to the 7-ketolithocholic acid (KLCA) of the formula (5)

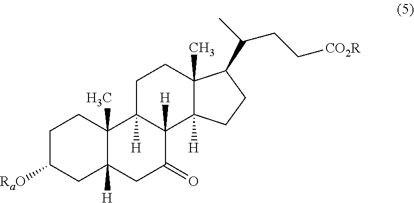

in which R and Rₐ have the meanings indicated above; and
d) KLCA of the formula (5) is reduced and, if Rₐ represents acyl, this acyl group is optionally removed, and
e) the reaction product is optionally further purified.

Here, if Rₐ represents acyl, this acyl group can be optionally removed after carrying out the reaction step b) or d).

Furthermore, the reaction of step a) can in particular take place in the presence of NAD(P)⁺.

Furthermore, NAD(P)⁺ consumed can be regenerated electrochemically or enzymatically in a manner known per se and/or the enzymes used can take place in immobilized form.

### 2. General Definitions

If no other details are given, the term “12α-HSDH” designates a dehydrogenase enzyme which catalyzes at least the stereospecific oxidation of cholic acid to 12-ketochenodeoxycholic acid with stoichiometric use of NAD⁺ or NADP⁺. The enzyme here can be a native or recombinantly produced enzyme. The enzyme can in principle be present in a mixture with cellular, such as, for example, protein impurities, but preferably in pure form.

A “pure form” or a “pure” or “essentially pure” enzyme is understood according to the invention as meaning an enzyme having a degree of purity of more than 80, preferably more than 90, in particular more than 95, and especially more than 99, % by weight, based on the total protein content, determined with the aid of customary protein detection methods, such as, for example, the biuret method or protein detection according to Lowry et al. (cf. description in R. K. Scopes, Protein Purification, Springer Verlag, New York, Heidelberg, Berlin (1982)). The specific activity of a 12α-HSDH enzyme according to the invention here is in the range indicated above.

A “redox equivalent” is understood as meaning a low molecular weight organic compound usable as an electron donor or electron acceptor, such as, for example, nicotinamide derivatives such as NAD⁺ and NADH⁺ or their reduced forms NADH or NADPH.

A compound of a special type, such as, for example, a “cholic acid compound” or an “ursodeoxycholic acid compound” is understood in particular as also meaning derivatives of the underlying starting compound (such as, for example, cholic acid or ursodeoxycholic acid).

Such derivatives comprise “salts”, such as, for example, alkali metal salts such as lithium, sodium and potassium salts of the compounds; and also ammonium salts, where an ammonium salt comprises the NH₄⁺ salt or those ammonium salts in which at least one hydrogen atom can be replaced by a C₁-C₆-alkyl radical. Typical alkyl radicals are, in particular, C₁-C₄-alkyl radicals, such as methyl, ethyl, n- or i-propyl, n-, sec- or tert-butyl, and n-pentyl and n-hexyl and the singly or multiply branched analogs thereof.

“Alkyl esters” of compounds according to the invention are in particular low-alkyl esters, such as, for example, C₁-C₆-alkyl esters. Nonlimiting examples which may be mentioned are methyl, ethyl, n- or i-propyl, n-, sec- or tert-butyl esters, or longer-chain esters, such as, for example, n-pentyl and n-hexyl esters and the singly or multiply branched analogs thereof.

“Amides” are in particular reaction products of acids according to the invention with ammonia or primary or secondary monoamines. Such amines are, for example, mono- or di-C₁-C₆-alkyl monoamines, where the alkyl radicals can optionally be further substituted independently of one another, such as, for example, by carboxyl, hydroxyl, halogen (such as F, Cl, Br, I), nitro and sulfonate groups.

“Acyl groups” according to the invention are in particular nonaromatic groups having 2 to 4 carbon atoms, such as, for example, acetyl, propionyl and butyryl, and aromatic groups having an optionally substituted mononuclear aromatic ring, where suitable substituents, for example, are selected from hydroxyl, halogen (such as F, Cl, Br, I), nitro and C₁-C₆-alkyl groups, such as, for example, benzoyl or toluoyl.

The hydroxysteroid compounds employed or produced according to the invention, such as cholic acid, glycocholic acid, taurocholic acid, ursodeoxycholic acid, 12-ketochenodeoxycholic acid, chenodeoxycholic acid and 7-ketolithocholic acid, can be employed in the process according to the invention or obtained therefrom in stereoisomerically pure pure form or in a mixture with other stereoisomers. Preferably, however, the compounds employed or produced are employed and/or isolated in essentially stereoisomerically pure form.

An “immobilization” is understood according to the invention as meaning the covalent or noncovalent binding of a biocatalyst used according to the invention, such as, for example, of a 12α-HSDH, to a solid carrier material, i.e. one essentially insoluble in the surrounding liquid medium.

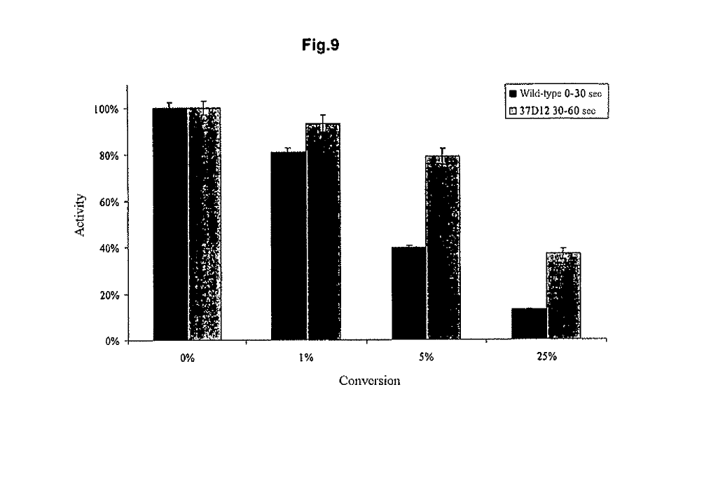

“Product inhibition” of the 12α-HSDH describes the reduction of the enzymatic activity in the presence of a product formed in an enzymatic reaction catalyzed by the enzyme. In the case of reaction to give CA, for example, inhibition by 12-keto-CDCA is thus to be observed. A “reduction of product inhibition” describes the reduced percentage decrease in the enzyme activity of an enzyme mutant according to the invention in comparison to a reference system, such as, for example, the native HSDH enzyme, in each case relative to the enzyme activity as a 100% activity value determined at 0% conversion (corresponding to 5 mM CA). This reduction can be determined as described in the experimental section, or in the legend to FIG. 9. Reductions of product inhibition according to the invention can also be expressed by means of the ratio of the residual activity of mutant to reference system in each case determined at the same percentage conversion. Thus the mutant according to the invention can have an activity increased by the factor 1.1 to 100, such as, for example, 1.5 to 20 or 2 to 10.

### 3. Further Embodiments of the Invention

### 3.1 Proteins

The present invention is not restricted to the proteins and/or enzymes with 12α-HSDH activity actually disclosed, but on the contrary also extends to functional equivalents thereof.

In the context of the present invention, “functional equivalents” or analogs of the enzymes actually disclosed are polypeptides different therefrom, which furthermore have the desired biological activity, such as, for example, 12α-HSDH activity.

Thus “functional equivalents” are understood as meaning, for example, enzymes that in the test used for 12α-HSDH activity have an around at least 1%, such as, for example, at least 10% or 20%, such as, for example, at least 50% or 75% or 90%, higher or lower activity of an enzyme comprising an amino acid sequence defined herein. Functional equivalents are moreover preferably stable between pH 4 to 11 and advantageously have a pH optimum in a range from pH 6 to 10, such as, in particular, 8.5 to 9.5, and a temperature optimum in the range from 15° C. to 80° C. or 20° C. to 70° C., such as, for example, approximately 45 to 60° C. or approximately 50 to 55° C.

The 12α-HSDH activity can be detected with the aid of various known tests. Without being restricted thereto, a test using a reference substrate, such as, for example, cholic acid, under standardized conditions as defined in the experimental section may be mentioned.

“Functional equivalents” are understood according to the invention, in particular, as also meaning “mutants” which in at least one sequence position of the above-mentioned amino acid sequences contain an amino acid other than that actually mentioned but nevertheless have one of the abovementioned biological activities. “Functional equivalents” thus comprise the mutants obtainable by one or more amino acid additions, substitutions, deletions and/or inversions, where the changes mentioned can occur in any sequence position, as long as they lead to a mutant with the property profile according to the invention. Functional equivalence is in particular also afforded if the reactivity patterns between mutant and unchanged polypeptide agree qualitatively, i.e., for example, identical substrates are converted with a different rate. Examples of suitable amino acid substitutions are summarized in the following table:

| Original residue | Examples of substitution |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the described polypeptides and “functional derivatives” and “salts” of the poly-peptides.

“Precursors” here are natural or synthetic precursors of the polypeptides with or without the desired biological activity.

The expression “salts” is understood as meaning both salts of carboxyl groups and acid addition salts of amino groups of the protein molecules according to the invention. Salts of carboxyl groups can be prepared in a manner known per se and comprise inorganic salts, such as, for example, sodium, calcium, ammonium, iron and zinc salts, and salts with organic bases, such as, for example, amines, such as triethanolamine, arginine, lysine, piperidine and the like. The invention likewise relates to acid addition salts, such as, for example, salts with mineral acids, such as hydrochloric acid or sulfuric acid, and salts with organic acids, such as acetic acid and oxalic acid.

“Functional derivatives” of polypeptides according to the invention can likewise be produced on functional amino acid side groups or on their N- or C-terminal end with the aid of known techniques. Such derivatives comprise, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups.

“Functional equivalents” of course also comprise polypeptides which are accessible from other organisms, and naturally occurring variants. For example, ranges of homologous sequence regions can be established by sequence comparison and equivalent enzymes can be determined following the precise specifications of the invention.

“Functional equivalents” likewise comprise fragments, preferably individual domains or sequence motifs, of the polypeptides according to the invention, which, for example, have the desired biological function.

“Functional equivalents” are moreover fusion proteins which contain one of the abovementioned polypeptide sequences or functional equivalents derived therefrom and at least one further heterologous sequence, functionally different therefrom, in a functional N- or C-terminal linkage (i.e. without mutual significant, functional impairment of the fusion protein parts). Nonlimiting examples of such heterologous sequences are, for example, signal peptides, histidine anchors or enzymes.

“Functional equivalents” additionally comprised according to the invention are homologs to the proteins actually disclosed. These have at least 60%, preferably at least 75%, in particular at least 85%, such as, for example, 90, 91, 92, 93, 94, 95, 96, 97, 98 or 99%, homology (or identity) to one of the amino acid sequences actually disclosed, calculated according to the algorithm of Pearson and Lipman, Proc. Natl. Acad. Sci. (USA) 85(8), 1988, 2444-2448. A percentage homology or identity of a homologous polypeptide according to the invention in particular means percentage identity of the amino acid residues relative to the total length of one of the amino acid sequences actually described herein.

The percentage identity values can also be determined by means of BLAST alignments, algorithm blastp (protein-protein BLAST), or by use of the Clustal adjustments indicated below.

In the case of a possible protein glycosylation, “functional equivalents” according to the invention comprise proteins of the type designated above in deglycosylated or glycosylated form and modified forms obtainable by alteration of the glycosylation pattern.

Homologs of the proteins or polypeptides according to the invention can be produced by mutagenesis, e.g. by point mutation, elongation or truncation of the protein.

Homologs of the proteins according to the invention can be identified by screening of combinatorial banks of mutants, such as, for example, truncation mutants. For example, a variegated bank of protein variants can be produced by combinatorial mutagenesis at the nucleic acid level, such as, for example, by enzymatic ligation of a mixture of synthetic oligonucleotides. There are a multiplicity of processes that can be used for the production of banks of potential homologs from a degenerate oligonucleotide sequence. The chemical synthesis of a degenerate gene sequence can be carried out in a DNA synthesizer, and the synthetic gene can then be ligated into a suitable expression vector. The use of a degenerate set of genes makes possible the preparation of all sequences in a mixture that encode the desired set of potential protein sequences. Processes for the synthesis of degenerate oligo-nucleotides are known to the person skilled in the art (e.g. Narang, S. A. (1983) Tetrahedron 39:3; Itakura et al. (1984) Annu. Rev. Biochem. 53:323; Itakura et al. (1984) Science 198:1056; Ike et al. (1983) Nucleic Acids Res. 11:477).

Several techniques for the screening of gene products of combinatorial banks that have been produced by point mutations or truncation, and for the screening of cDNA banks for gene products with a selected property, are known in the prior art. These techniques can be adapted to the rapid screening of the gene banks that have been produced by combinatorial mutagenesis of homologs according to the invention. The most frequently used techniques for the screening of large gene banks that undergo an analysis with a high throughput comprise cloning of the gene bank into replicable expression vectors, transformation of the suitable cells with the resulting vector bank and expression of the combinatorial genes under conditions under which the detection of the desired activity facilitates the isolation of the vector which encodes the gene whose product was detected. Recursive-ensemble mutagenesis (REM), a technique that increases the frequency of functional mutants in the banks, can be used in combination with the screening tests in order to identify homologs (Arkin and Yourvan (1992) PNAS 89:7811-7815; Delgrave et al. (1993) Protein Engineering 6(3): 327-331).

### 3.2 Nucleic Acids and Constructs

### 3.2.1 Nucleic Acids

The invention also relates to nucleic acid sequences that code for an enzyme with 12α-HSDH activity.

The present invention also relates to nucleic acids with a certain degree of identity to the actual sequences described herein.

“Identity” between two nucleic acids is understood as meaning the identity of the nucleotides over the total nucleic acid length in each case, in particular the identity that is calculated by comparison with the aid of the Vector NTI Suite 7.1 software of the company Informax (USA) using the Clustal Method (Higgins D G, Sharp P M. Fast and sensitive multiple sequence alignments on a microcomputer. Comput Appl. Biosci. 1989 April; 5(2): 151-1) with adjustment of the following parameters:

### Multiple Alignment Parameters:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weighing | 0 |

### Pairwise Alignment Parameter:

| FAST algorithm | on |
|---|---|
| K-tuple size | 1 |
| Gap penalty | 3 |
| Window size | 5 |
| Number of best diagonals | 5 |

Alternatively to this, the identity can also be determined according to Chenna, Ramu, Sugawara, Hideaki, Koike, Tadashi, Lopez, Rodrigo, Gibson, Toby J, Higgins, Desmond G, Thompson, Julie D. Multiple sequence alignment with the Clustal series of programs. (2003) Nucleic-Acids Res 31 (13): 3497-500, according to Internet address: http://www.ebi. ac.uk/Tools/clustalw/index.html# and with the following parameters:

| | |
|---|---|
| DNA Gap Open Penalty | 15.0 |
| DNA Gap Extension Penalty | 6.66 |
| DNA Matrix | Identity |
| Protein Gap Open Penalty | 10.0 |
| Protein Gap Extension Penalty | 0.2 |
| Protein matrix | Gonnet |
| Protein/DNA ENDGAP | −1 |
| Protein/DNA GAPDIST | 4 |

All nucleic acid sequences mentioned herein (single- and double-stranded DNA and RNA sequences, such as, for example, cDNA and mRNA) can be produced in a manner known per se by chemical synthesis from the nucleotide structural units, such as, for example, by fragment condensation of individual overlapping, complementary nucleic acid structural units of the double helix. The chemical synthesis of oligonucleotides can be carried out, for example, in a known manner, according to the phosphoamidite method (Voet, Voet, 2nd edition, Wiley Press New York, pages 896-897). The addition of synthetic oligonucleotides and the filling of gaps with the aid of the Klenow fragment of the DNA polymerase and ligation reactions and general cloning processes are described in Sambrook et al. (1989), Molecular Cloning: A laboratory manual, Cold Spring Harbor Laboratory Press.

The invention also relates to nucleic acid sequences (single- and double-stranded DNA and RNA sequences, such as, for example, cDNA and mRNA), coding for one of the above polypeptides and their functional equivalents, which are accessible, for example, using synthetic nucleotide analogs.

The invention relates both to isolated nucleic acid molecules, which code for polypeptides and proteins according to the invention or biologically active sections thereof, and nucleic acid fragments that can be used, for example, for use as hybridization probes or primers for the identification or amplification of encoding nucleic acids according to the invention.

The nucleic acid molecules according to the invention can moreover contain untranslated sequences of the 3′- and/or 5′-end of the encoding gene region.

The invention furthermore comprises the nucleic acid molecules or a section thereof complementary to the nucleotide sequences actually described.

The nucleotide sequences according to the invention make possible the production of probes and primers that can be used for the identification and/or cloning of homologous sequences in other cell types and organisms. Such probes or primers usually comprise a nucleotide sequence region that hybridizes under “stringent” conditions (see below) on at least approximately 12, preferably at least approximately 25, such as, for example, approximately 40, 50 or 75, successive nucleotides of a sense strand of a nucleic acid sequence according to the invention or of a corresponding antisense strand.

An “isolated” nucleic acid molecule is separated from other nucleic acid molecules that are present in the natural source of the nucleic acid and can moreover be essentially free of other cellular material or culture medium if it is produced by recombinant techniques, or be free of chemical precursors or other chemicals if it is chemically synthesized.

A nucleic acid molecule according to the invention can be isolated by means of molecular biological standard techniques and the sequence information made available according to the invention. For example, cDNA can be isolated from a suitable cDNA bank by using one of the completed sequences actually disclosed or a section thereof as a hybridization probe and standard hybridization techniques (as described, for example, in Sambrook, J., Fritsch, E. F. and Maniatis, T. Molecular Cloning: A Laboratory Manual. 2nd ed., Cold Spring Harbor Laboratory, Cold Spring Harbor Laboratory Press, Cold Spring Harbor, N.Y., 1989). Moreover, a nucleic acid molecule comprising one of the disclosed sequences or a section thereof can be isolated by polymerase chain reaction, the oligonucleotide primers that were prepared on the basis of this sequence being used. The nucleic acid amplified in this way can be cloned into a suitable vector and characterized by DNA sequence analysis. The oligonucleotides according to the invention can furthermore be produced by standard synthesis processes, e.g. with an automatic DNA synthesizer.

Nucleic acid sequences according to the invention or derivatives thereof, homologs or parts of these sequences can be isolated from other bacteria, for example, using customary hybridization processes or the PCR technique, e.g. by means of genomic or cDNA banks. These DNA sequences hybridize under standard conditions with the sequences according to the invention.

“Hybridize” is understood as meaning the ability of a poly- or oligonucleotide to bind to an almost complementary sequence under standard conditions, while nonspecific bonds between noncomplementary partners are suppressed under these conditions. For this, the sequences can be complementary to 90-100%. The property of complementary sequences to be able to bind specifically to one another is made use of, for example, in the Northern or Southern Blot technique or in primer binding in PCR or RT-PCR.

For hybridization, short oligonucleotides of the conserved regions are advantageously used. It is also possible, however, to use longer fragments of the nucleic acids according to the invention or the complete sequences for the hybridization. These standard conditions vary according to the nucleic acid used (oligonucleotide, longer fragment or complete sequence) or depending on which nucleic acid type DNA or RNA are used for the hybridization. Thus, for example, the melting temperatures for DNA:DNA hybrids are about 10° C. lower than those of DNA:RNA hybrids of identical length.

Standard conditions are understood as meaning, for example, according to nucleic acid, temperatures between 42 and 58° C. in an aqueous buffer solution with a concentration of between 0.1 to 5×SSC (1×SSC=0.15 M NaCl, 15 mM sodium citrate, pH 7.2) or additionally in the presence of 50% formamide such as, for example, 42° C. in 5×SSC, 50% formamide. Advantageously, the hybridization conditions for DNA: DNA hybrids are 0.1×SSC and temperatures between approximately 20° C. and 45° C., preferably between approximately 30° C. and 45° C. For DNA:RNA hybrids, the hybridization conditions are advantageously 0.1×SSC and temperatures between approximately 30° C. and 55° C., preferably between approximately 45° C. and 55° C. These specified temperatures for the hybridization are, by way of example, calculated melting temperature values for a nucleic acid with a length of about 100 nucleotides and a G+C content of 50% in the absence of formamide. The experimental conditions for the DNA hybridization are described in relevant textbooks of genetics, such as, for example, Sambrook et al., “Molecular Cloning”, Cold Spring Harbor Laboratory, 1989, and can be calculated according to formulae known to the person skilled in the art, for example, depending on the length of the nucleic acids, the nature of the hybrids or the G+C content. The person skilled in the art can infer further information on hybridization from the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The “hybridization” can in particular be carried out under stringent conditions. Such hybridization conditions are described, for example, in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

“Stringent” hybridization conditions are to be understood as meaning in particular: Incubation at 42° C. overnight in a solution consisting of 50% of formamide, 5×SSC (750 mM NaCl, 75 mM trisodium citrate), 50 mM sodium phosphate (pH 7.6), 5×Denhardt solution, 10% dextran sulfate and 20 g/ml of denatured, sheared salmon sperm DNA, followed by a washing step of the filters with 0.1×SSC at 65° C.

The invention also relates to derivatives of the actually disclosed or derivable nucleic acid sequences.

Thus further nucleic acid sequences according to the invention can be derived, for example, from SEQ ID NO: 1 or 3 and differ therefrom by addition, substitution, insertion or deletion of single or multiple nucleotides, but furthermore code for polypeptides with the desired property profile.

Also comprised according to the invention are those nucleic acid sequences that comprise “blunt” mutations or are modified corresponding to the codon utilization of a special origin or host organism, in comparison to an actually mentioned sequence, as well as naturally occurring variants, such as, for example, splice variants or allele variants, thereof.

The invention likewise relates to sequences obtainable by conservative nucleotide substitutions (i.e. the amino acid concerned is replaced by an amino acid of identical charge, size, polarity and/or solubility).

The invention also relates to the molecules derived by sequence polymorphisms of the nucleic acids actually disclosed. These genetic polymorphisms can exist between individuals within a population on account of the natural variation. These natural variations usually cause a variance of 1 to 5% in the nucleotide sequence of a gene.

Derivatives of the nucleic acid sequence according to the invention with the sequence SEQ ID NO: 1 or 3 are to be understood as meaning, for example, allele variants that have at least 60% homology at the derived amino acid level, preferably at least 80% homology, very particularly preferably at least 90% homology, over the entire sequence range (with respect to homology at amino acid level, reference may be made to the above remarks for the polypeptides). Over partial regions of the sequences, the homologies can advantageously be higher.

Furthermore, derivatives are also to be understood as meaning homologs of the nucleic acid sequences according to the invention, in particular of the SEQ ID NO: 1 and 3, for example fungal or bacterial homologs, truncated sequences, single-strand DNA or RNA of the encoding and nonencoding DNA sequence. Thus, for example, homologs for SEQ ID NO: 7 at the DNA level have a homology of at least 40%, preferably of at least 60%, particularly preferably of at least 70%, very particularly preferably of at least 80% over the entire DNA range indicated in SEQ ID NO: 7.

Moreover, derivatives are understood as meaning, for example, fusions with promoters. The promoters that are added before the nucleotide sequences indicated can be modified by at least one nucleotide exchange, at least one insertion, inversion and/or deletion without the functionality or activity of the promoters, however, being adversely affected. In addition, the promoters can be increased in their activity by modification of their sequence or also replaced completely by more active promoters of foreign organisms.

Processes for the production of functional mutants are moreover known to the person skilled in the art.

According to the technique used, the person skilled in the art can introduce completely random or alternatively more specific mutations into genes or alternatively nonencoding nucleic acid regions (which are important, for example, for regulation of the expression) and subsequently prepare gene banks. The molecular biological methods necessary for this are known to the person skilled in the art and described, for example, in Sambrook and Russell, Molecular Cloning, 3rd Edition, Cold Spring Harbor Laboratory Press 2001.

Methods for the modification of genes and thus for the modification of the proteins encoded by these have been familiar to the person skilled in the art for a long time, such as, for example,
site-specific mutagenesis, in which single or multiple nucleotides of a gene are specifically replaced (Trower M K (editor) 1996; In vitro mutagenesis protocols. Humana Press, New Jersey),
saturation mutagenesis, in which a codon for any desired amino acid can be replaced or added in any desired site of a gene (Kegler-Ebo D M, Docktor C M, DiMaio D (1994) Nucleic Acids Res 22:1593; Barettino D, Feigenbutz M, Valcárel R, Stunnenberg H G (1994) Nucleic Acids Res 22:541; Barik S (1995) Mol Biotechnol 3:1),
error-prone polymerase chain reaction (error-prone PCR), in which nucleoside sequences are mutated by erroneously working DNA polymerases (Eckert K A, Kunkel T A (1990) Nucleic Acids Res 18:3739);
the passaging of genes to mutator strains, in which, for example, an increased mutation rate of nucleotide sequences occurs on account of defective DNA repair mechanisms (Greener A, Callahan M, Jerpseth B (1996) An efficient random mutagenesis technique using an E. coli mutator strain, In: Trower M K (editor) In vitro mutagenesis protocols. Humana Press, New Jersey), or
DNA shuffling, in which a pool of closely related genes is formed and digested and the fragments are used as templates for a polymerase chain reaction, in which mosaic genes of full length are finally produced by repeated strand separation and reapproximation (Stemmer W P C (1994) Nature 370:389; Stemmer W P C (1994) Proc Natl Acad Sci USA 91:10747).

Using “directed evolution” (described, inter alia, in Reetz M T and Jaeger K-E (1999), Topics Curr Chem 200:31; Zhao H, Moore J C, Volkov A A, Arnold F H (1999), Methods for optimizing industrial enzymes by directed evolution, in: Demain A L, Davies J E (eds.) Manual of industrial microbiology and biotechnology, American Society for Microbiology), the person skilled in the art can also produce functional mutants in a selective manner and also on a large-scale. Here, in a first step gene banks of the respective proteins are initially produced, the methods indicated above, for example, being able to be used. The gene banks are expressed in a suitable manner, for example by bacteria or by phage display systems.

The concerned genes of host organisms that express functional mutants with properties which largely correspond to the desired properties can be subjected to a further round of mutation. The steps of mutation and of selection or of screening can be repeated iteratively as long as the present functional mutants have the desired properties in adequate measure. As a result of this iterative procedure, a limited number of mutations, such as, for example, 1 to 5 mutations, can be performed stepwise and assessed and selected for their influence on the enzyme property concerned. The selected mutant can then be subjected to a further mutation step in the same manner. The number of individual mutants to be investigated can be significantly decreased thereby.

The results according to the invention yield important information with respect to structure and sequence of the enzymes concerned, which are necessary in order specifically to generate further enzymes with desired modified properties. In particular, “hot spots” can be defined, i.e. sequence sections that are potentially suitable for modifying an enzyme property by means of the introduction of specific mutations.

Nonlimiting examples of such hot-spot regions of the HSDH according to the invention are summarized below:

35-40, in particular 37-38, (in each case relative to the amino acid sequence of HSDH_short (SEQ ID NO: 4).

90-105, 93-100 or 96-100, in particular 97 and/or 98, (in each case relative to the amino acid sequence of HSDH_short (SEQ ID NO: 4).

### 3.2.2 Constructs

The invention moreover relates to expression constructs comprising a nucleic acid sequence coding for a polypeptide according to the invention under the genetic control of regulative nucleic acid sequences; and vectors comprising at least one of these expression constructs.

An “expression unit” is understood according to the invention as meaning a nucleic acid with expression activity, which comprises a promoter, as defined herein, and after functional linkage with a nucleic acid to be expressed or a gene, regulates the expression, that is the transcription and the translation of this nucleic acid or this gene. Therefore also spoken of in this connection is a “regulative nucleic acid sequence”. In addition to the promoter, further, regulative elements, such as, for example, enhancers, can be present.

An “expression cassette” or “expression construct” is understood according to the invention as meaning an expression unit that is functionally linked with the nucleic acid to be expressed or the gene to be expressed. In contrast to an expression unit, an expression cassette thus comprises not only nucleic acid sequences which regulate transcription and translation, but also the nucleic acid sequences which are to be expressed as protein as a consequence of the transcription and translation.

In the context of the invention, the terms “expression” or “overexpression” describe the production and increase in the intracellular activity of one or more enzymes in a microorganism, which are encoded by the corresponding DNA. To this end, for example, a gene can be introduced into an organism, a gene present can be replaced by another gene, the copy number of the gene or of the genes can be increased, a strong promoter can be used or a gene can be used that codes for a corresponding enzyme with a high activity and these measures can optionally be combined.

Preferably, such constructs according to the invention comprise a promoter 5′-upstream of the respective encoding sequence and a terminator sequence 3′-downstream, and optionally further customary regulative elements, namely in each case operatively linked with the encoding sequence.

A “promoter”, a “nucleic acid with promoter activity” or a “promoter sequence” is understood according to the invention as meaning a nucleic acid that regulates the transcription of nucleic acid in functional linkage with a nucleic acid to be transcribed.

A “functional” or “operative” linkage is understood in this connection, for example, as meaning the sequential arrangement of one of the nucleic acids with promoter activity and of a nucleic acid sequence to be transcribed and optionally further regulative elements, such as, for example, nucleic acid sequences that guarantee the transcription of nucleic acids, and, for example, a terminator, such that each of the regulative elements can fulfill its function in the transcription of the nucleic acid sequence. To this end, a direct linkage in the chemical sense is not absolutely necessary. Genetic control sequences, such as, for example, enhancer sequences, can also exert their function from on the target sequence from further removed positions or even from other DNA molecules.

Arrangements are preferred in which the nucleic acid sequence to be transcribed is positioned behind (i.e. at the 3′-end of) the promoter sequence, so that both sequences are covalently linked with one another. Here, the distance between the promoter sequence and the nucleic acid sequence to be expressed transgenically can be less than 200 base pairs, or smaller than 100 base pairs or smaller than 50 base pairs.

In addition to promoters and terminator, examples of further regulative elements which may be mentioned are targeting sequences, enhancers, polyadenylation signals, selectable markers, amplification signals, replication origins and the like. Suitable regulatory sequences are described, for example, in Goeddel, Gene Expression Technology: Methods in Enzymology 185, Academic Press, San Diego, Calif. (1990).

Nucleic acid constructs according to the invention in particular comprise sequence SEQ ID NO: 1 or 3 or derivatives and homologs thereof, and the nucleic acid sequences derivable therefrom, which were operatively or functionally linked advantageously with one or more regulation signals for the control, e.g. increase, of gene expression.

Additionally to these regulation sequences, the natural regulation of these sequences can additionally be present before the actual structural genes and can have optionally been genetically modified such that the natural regulation has been switched off and the expression of the genes increased. The nucleic acid construct, however, can also be constructed more simply, that is no additional regulation signals have been inserted before the encoding sequence and the natural promoter with its regulation has not been removed. Instead of this, the natural regulation sequence is mutated such that regulation no longer takes place and the gene expression is increased.

A preferred nucleic acid construct advantageously also contains one or more of the already mentioned “enhancer” sequences, functionally linked with the promoter, which make possible increased expression of the nucleic acid sequence. Additional advantageous sequences can also be inserted at the 3′-end of the DNA sequences, such as further regulatory elements or terminators. The nucleic acids according to the invention can be present in the construct in one or more copies. Still further markers can be present in the construct, such as genes complementing antibiotic resistances or auxotrophies, optionally for selection on the construct.

Examples of suitable regulation sequences are present in promoters such as cos, tac, trp, tet, trp-tet, lpp, lac, lpp-lac, lacl^{q}, T7, T5, T3, gal, trc, ara, rhaP (rhaP_{BAD})SP6, lambda-P_{R} or in the lambda-P_{T}, promoter, which are advantageously used in gram-negative bacteria. Further advantageous regulation sequences are present, for example, in the gram-positive promoters amy and SPO2, in the yeast or fungal promoters ADC1, MFalpha, AC, P-60, CYC1, GAPDH, TEF, rp28 and ADH. Artificial promoters can also be used for the regulation.

For expression in a host organism, the nucleic acid construct is advantageously inserted into a vector, such as, for example, a plasmid or a phage that makes possible optimal expression of the genes in the host. Apart from plasmids and phages, vectors are also understood as meaning all other vectors known to the person skilled in the art, that is, for example, viruses, such as SV40, CMV, baculovirus and adenovirus, transposons, IS elements, phasmids, cosmids, and linear or circular DNA. These vectors can be replicated autonomously in the host organism or replicated chromosomally. These vectors represent a further embodiment of the invention.

Suitable plasmids are, for example, pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, λgt11 or pBdCI in E. coli, pIJ101, pIJ364, pIJ702 or pIJ361 in Streptomyces, pUB110, pC194 or pBD214 in Bacillus, pSA77 or pAJ667 in Corynebacterium, pALS1, pIL2 or pBB116 in fungi, 2alphaM, pAG1, YEp6, YEp13 or pEMBLYe23 in yeasts or pLGV23, pGHIac⁺, pBIN19, pAK2004 or pDH51 in plants. The plasmids mentioned are a small selection of the possible plasmids. Further plasmids are well known to the person skilled in the art and can be inferred, for example, from the book Cloning Vectors (Eds. Pouwels P. H. et al. Elsevier, Amsterdam-New York-Oxford, 1985, ISBN 0 444 904018).

In a further embodiment of the vector, the vector containing the nucleic acid construct according to the invention or the nucleic acid according to the invention can also advantageously be introduced into the microorganisms in the form of a linear DNA and integrated into the genome of the host organism by means of heterologous or homologous recombination. This linear DNA can consist of a linearized vector such as a plasmid or only of the nucleic acid construct or the nucleic acid according to the invention.

For optimal expression of heterologous genes in organisms, it is advantageous to modify the nucleic acid sequences corresponding to the specific “codon utilization” used in the organism. The “codon utilization” can easily be determined by means of computer analyses of other, known genes of the organism concerned.

The production of an expression cassette according to the invention is carried out by fusion of a suitable promoter with a suitable encoding nucleotide sequence and a terminator or polyadenylation signal. To this end, customary recombination and cloning techniques are used, such as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector that makes possible an optimal expression of the genes in the host. Vectors are well known to the person skilled in the art and can be inferred, for example, from “Cloning Vectors” (Pouwels P. H. et al., editor, Elsevier, Amsterdam-New York-Oxford, 1985).

### 3.3 Microorganisms

Depending on context, the term “microorganism” can be understood as meaning the starting microorganism (wild-type) or a genetically modified, recombinant microorganism or both.

With the aid of the vectors according to the invention, recombinant microorganisms can be prepared which are transformed, for example, with at least one vector according to the invention and can be employed for the production of the polypeptides according to the invention. Advantageously, the recombinant constructs according to the invention described above are introduced into a suitable host system and expressed. Here, familiar cloning and transfection methods known to the person skilled in the art are preferably used, such as, for example, coprecipitation, protoplast fusion, electroporation, retroviral transfection and the like, in order to express the nucleic acids mentioned in the respective expression system. Suitable systems are described, for example, in Current Protocols in Molecular Biology, F. Ausubel et al., editor, Wiley Interscience, New York 1997, or Sambrook et al. Molecular Cloning: A Laboratory Manual, 2nd ed., Cold Spring Harbor Laboratory, Cold Spring Harbor Laboratory Press, Cold Spring Harbor, N.Y., 1989.

Possible recombinant host organisms for the nucleic acid according to the invention or the nucleic acid construct are in principle all prokaryotic or eukaryotic organisms. Advantageously, the host organisms used are microorganisms such as bacteria, fungi or yeasts. Gram-positive or gram-negative bacteria are advantageously used, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium, Clostridium or Rhodococcus. The genus and species Escherichia coli is very particularly preferred. Further advantageous bacteria are moreover to be found in the group consisting of the alpha-proteobacteria, beta-proteobacteria or gamma-proteobacteria.

The host organism or the host organisms according to the invention here preferably contain at least one of the nucleic acid sequences, nucleic acid constructs or vectors described in this invention that code for an enzyme with 12α-HSDH activity according to the above definition.

The organisms used in the process according to the invention are grown or cultured in a manner known to the person skilled in the art according to the host organism. Microorganisms are generally grown in a liquid medium that contains a carbon source usually in the form of sugars, a nitrogen source usually in the form of organic nitrogen sources such as yeast extract or salts such as ammonium sulfate, trace elements such as iron, manganese or magnesium salts and optionally vitamins, at temperatures between 0° C. and 100° C., preferably between 10° C. and 60° C. with oxygen gassing. The pH of the nutrient liquid here can be kept at a fixed value, that is regulated or not during growth. Growth can take place batchwise, semi-batchwise or continuously. Nutrients can be introduced at the start of fermentation or subsequently fed semi-continuously or continuously.

### 3.4 Production of UDCA

### 3.4.1 Introduction

The active substances ursodeoxycholic acid (UDCA) and the associated diastereomer chenodeoxycholic acid (CDCA), inter alia, have been employed for many years for the medicinal treatment of cholelithiasis. Both compounds differ only by the configuration of the hydroxyl group on C atom 7 (UDCA: β-configuration, CDCA: α-configuration). For the production of commercial amounts of UDCA, a process has preferably been used hitherto in which CDCA is employed as a raw material. COCA in turn is preferably produced from cholic acid (CA).

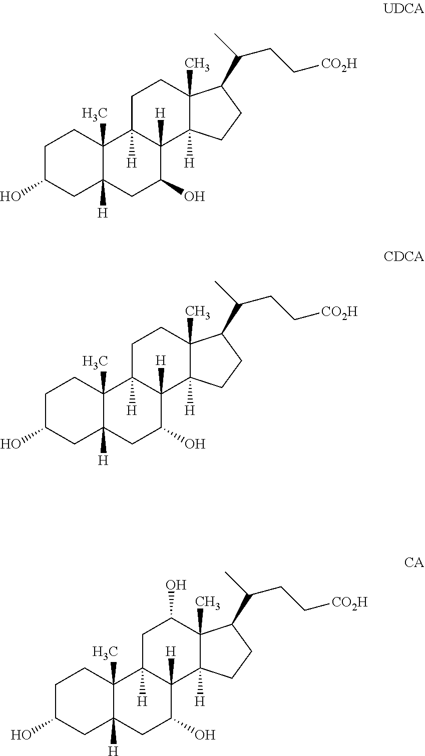

### 3.4.2 Production of CDCA

CA (CAS 81-25-4) is used as a raw material for the production of CDCA (CAS 474-25-9). The classical chemical route 1 makes use exclusively of chemical processing steps. In this case, four steps are necessary in order to convert CA to CDCA. The alternative route 2 comprises the enzyme-catalyzed reaction. This pathway leads from CA to CDCA in only two steps.

### 3.4.2.1 Route 1 (Chemical Pathway)

In the First Step of this Synthesis, the Carboxylic acid group of the CA is esterified to give the methyl ester (COCA I, CAS 1448-36-8). The regioselectively proceeding acetylation of the hydroxyl groups in positions 3 and 7 follows. The acetylation product, methyl 3,7-di-O-acetylcholate (CDCA II, CAS 3749-87-9) is obtained crystalline and is isolated. In the following stage (step 3), the free hydroxyl group in position 12 is oxidized. The methyl 3,7-di-O-acetyl-12-ketocholanate (CDCA III, CAS 4651-67-6) is deoxygenated to CDCA in the fourth and last step in a Wolff-Kishner reduction.

### 1st Step: Esterification

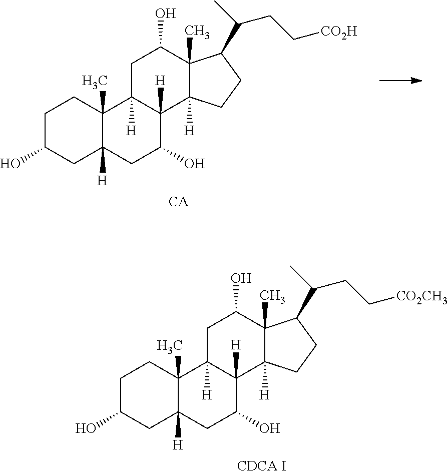

### 2nd Step: Acetylation

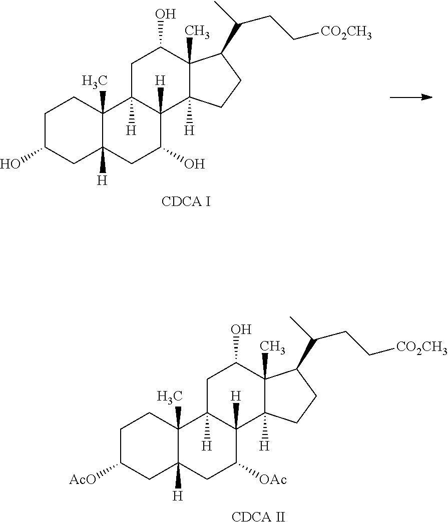

### 3rd Step: Oxidation

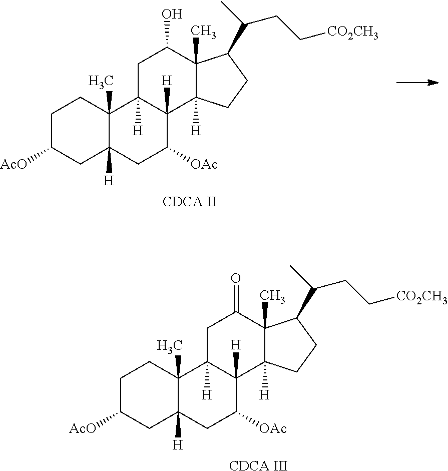

### 4th Step: Deoxygenation

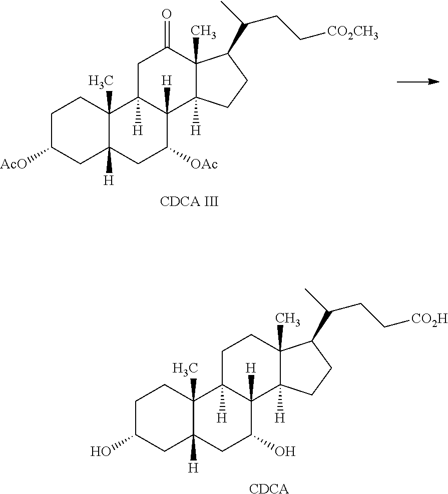

In detail, the process is carried out as follows:

In stage 1, CA is esterified with methanol under acid catalysis to give methyl cholate (CDCA I). Regio-selective acetylation of the hydroxyl groups in positions 3 and 7 with acetic anhydride follows. An organic nitrogen base and an acylation catalyst is optionally used for the reaction. By optimization of the reaction time, a maximum of the diacetyl compound (CDCA II) is achieved here. The product is isolated after crystallization and dried. Acetylation conditions, in particular the combination acetic anhydride, triethylamine and DMAP, are described in EP 0 424 232. The selectivity of the acetylation decides on the later quality of the (intermediate) product CDCA. The by-product methyl 3-O-monoacetylcholate leads in the further course of the synthesis to lithocholic acid. This is toxic and is limited in the monographs of the end product UDCA to a low value (Ph. Eur. 0.1%, USP 0.05%). In the case of an overacetylation to methyl 3,7,12-tri-O-acetylcholate, the COCA obtained later contains proportionately more CA as an impurity.

The oxidation of the CDCA II to CDCA III is carried out using aqueous sodium hypochlorite solution. The product precipitates from the reaction solution, and is filtered off and dried. This procedure also is described in EP 0 424 232. Generally, still other oxidants are found in the literature as alternatives, such as chromic acid.

For deoxygenation of the CDCA III to CDCA, various variants of the Wolff-Kishner reduction are known. In one method, COCA III is reacted with hydrazine and sodium hydroxide in triethylene glycol at 200° C. The product is precipitated from the reaction solution by acidifying with hydrochloric acid, and is subsequently filtered off and dried. Another method is described in EP 0 424 232 and works at lower temperature. CDCA III is reacted here with hydrazine and potassium hydroxide in 2-butanol. The product is precipitated from water as in variant 1 by addition of hydrochloric acid.

The CDCA obtained by this process has a defined and specified quality that is suitable in order to prepare UDCA in pharmacopeia quality by the process described later.

### 3.4.2.2 Route 2 (Enzymatic Pathway)

As an alternative to the exclusively chemical process, according to the invention an enzyme-catalyzed oxidation of CA to 12-ketochenodeoxycholic acid (12-keto-CDCA, CAS 2458-08-4), which is then reacted further to give CDCA, is provided. This synthesis pathway comprises only two steps and is thus clearly simpler to carry out in comparison to the purely chemical route.

### 1st Step: Enzymatic Oxidation

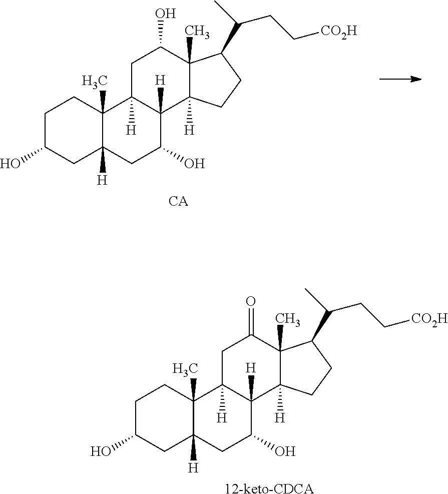

### Second Step: Deoxygenation

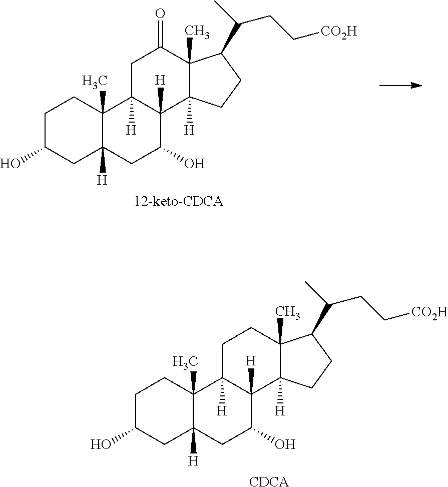

According to step 1, cholic acid is oxidized NADP⁺-dependently by means of 12α-HSDH to give 12-ketochenodeoxycholic acid (12-keto-CDCA). This reaction is reversible. 12α-HSDHs belong to enzyme class 1.1.1.176 and are mainly found in bacteria of the genus Clostridium. Both NADP⁺-dependent (Harris and Hylemon (1978) Biochim Biophys Acta 528(1): 148-57) and NAD⁺-dependent representatives exist (MacDonald at al. 1976) Biochim Biophys Acta 450(2): 142-53.

The only known microorganism that expresses a high 12α-HSDH activity in the absence of other HSDHs is Clostridium sp. group P strain 48-50 DSM 4029 (MacDonald at al. 1979, loc. cit.). Therefore this organism was hitherto employed as a producer of 12α-HSDH, a demanding, anaerobic fermentation with cost-intensive medium being necessary (MacDonald 1981) Experientia 37(5): 451-2. However, it was possible to replace the latter by yeast autolysate (Braun, M. et al. 1991, loc. cit.).

The enzymatic oxidation is carried out according to the invention preferably by means of a 12α-HSDH according to the invention (long or short version) and cofactor regeneration by means of an ADH, such as, for example, ADH ms or ADH t.

The deoxygenation according to step 2 is a classical chemical Wolff-Kishner reduction and is carried out analogously to the deoxygenation of CDCA III described above. An essential advantage of this route is that as a result of the selectivity of the enzyme the impurity lithocholic acid is not formed.

### 3.4.3.3 Production of UDCA

CDCA is used as a raw material for UDCA (CAS 128-13-2). In the first synthesis step, the hydroxyl group in position 7 of the CDCA is oxidized to give the corresponding ketone. 7-Ketolithocholic acid (3α-hydroxy-7-ketocholanic acid, in short: KLCA, CAS 4651-67-6) results. The stereoselective reduction of the keto group in position 7 follows in the second step. The aim is to obtain UDCA with as high diastereo-selectivity as possible. Generally, the UDCA directly after the reduction still contains a few percent of the diastereomer CDCA. In order to arrive at the active substance UDCA, crude UDCA must be purified in a third step.

### 1st Step: Oxidation

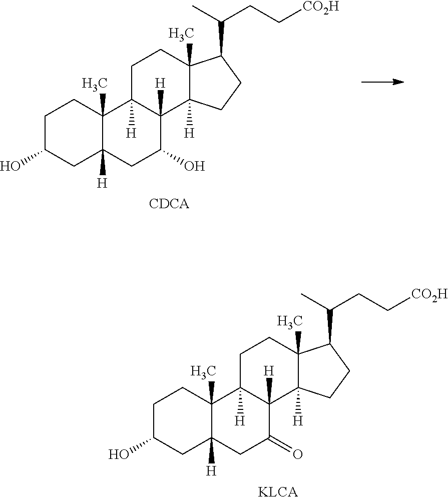

### 2nd Step: Reduction

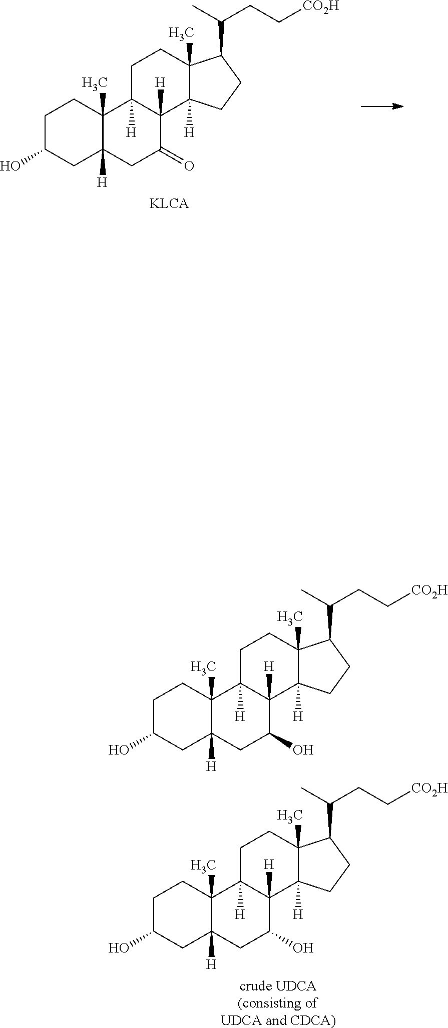

### 3rd Step: Purification

### Crude UDCA→Pure UDCA

The oxidation of the CDCA is customarily carried out with aqueous sodium hypochlorite solution. In the literature, chromic acid oxidation is additionally found as an alternative. KLCA is obtained as a solid that is then processed further in the second step. The reduction can be carried out with sodium metal in alcohols. A crude product results with a composition of UDCA:CDCA of about 85:15. In alternative processes, KLCA is reduced with hydrogen on a nickel catalyst (Raney nickel) in alcohols (such as, for example, aliphatic alcohols) as a solvent together with a base, such as potassium t-butoxide or potassium hydroxide (EP-A-0 230 085). Additionally, reduction with potassium and lithium (higher selectivity than sodium, C. Giordano et al. Angew. Chem. 1985, 97, 510) and zinc (ES 489661) and electrochemically (U.S. Pat. No. 4,547,271) is also possible.

The purification of crude UDCA to give pure UDCA involves a separation of diastereomeric salts. It is carried out by preparation, isolation and subsequent cleavage of a suitable salt of UDCA. The following alternative purification methods are mentioned in the literature: preparation, recrystallization and cleavage of a corresponding UDCA ester (EP-A-0 386 538), extractions (JP 60006699) and chromatographic processes (IT 2000MI1177).

### 3.5 Recombinant Production of 12α-HSDH

The invention furthermore relates to processes for the recombinant production of polypeptides according to the invention or functional, biologically active fragments thereof, in which a polypeptide-producing microorganism is cultured, the expression of the polypeptides is optionally induced and these are isolated from the culture. The polypeptides can thus also be produced on the industrial scale, if this is desired.

The microorganisms produced according to the invention can be cultured continuously or discontinuously in the batch process (batch culturing) or in the fed batch or repeated fed batch process. A summary of known culturing methods is to be found in the textbook of Chmiel (Bioprozeβtechnik 1. Einführung in die Bioverfahrenstechnik [Bioprocess technology 1. Introduction to bioprocess technology] (Gustav Fischer Verlag, Stuttgart, 1991)) or in the textbook of Storhas (Bioreaktoren and periphere Einrichtungen [Bioreactors and peripheral devices] (Vieweg Verlag, Brunswick/Wiesbaden, 1994)).

The culture medium to be used has to suitably meet the demands of the respective strains. Descriptions of culture media of various microorganisms are contained in the Handbook “Manual of Methods for General Bacteriology” of the American Society for Bacteriology (Washington D.C., USA, 1981).

These media, which can be employed according to the invention, usually comprise one or more carbon sources, nitrogen sources, inorganic salts, vitamins and/or trace elements.

Preferred carbon sources are sugars, such as mono-, di- or polysaccharides. Very good carbon sources are, for example, glucose, fructose, mannose, galactose, ribose, sorbose, ribulose, lactose, maltose, sucrose, raffinose, starch or cellulose. Sugars can also be added to the media by means of complex compounds, such as molasses, or other by-products of sugar refining. It can also be advantageous to add mixtures of various carbon sources. Other possible carbon sources are oils and fats such as, for example, soybean oil, sunflower oil, peanut oil and coconut oil, fatty acids such as, for example, palmitic acid, stearic acid or linoleic acid, alcohols such as, for example, glycerol, methanol or ethanol and organic acids such as, for example, acetic acid or lactic acid.

Nitrogen sources are usually organic or inorganic nitrogen compounds or materials that contain these compounds. Exemplary nitrogen sources comprise ammonia gas or ammonium salts, such as ammonium sulfate, ammonium chloride, ammonium phosphate, ammonium carbonate or ammonium nitrate, nitrates, urea, amino acids or complex nitrogen sources, such as corn-steep liquor, soybean flour, soy protein, yeast extract, meat extract and others. The nitrogen sources can be used individually or as a mixture.

Inorganic salt compounds that can be present in the media comprise the chloride, phosphorus or sulfate salts of calcium, magnesium, sodium, cobalt, molybdenum, potassium, manganese, zinc, copper and iron.

As a sulfur source, it is possible to use inorganic sulfur-containing compounds such as, for example, sulfates, sulfites, dithionites, tetrathionates, thiosulfates, sulfides but also organic sulfur compounds, such as mercaptans and thiols.

As a phosphorus source, it is possible to use phosphoric acid, potassium dihydrogenphosphate or dipotassium hydrogenphosphate or the corresponding sodium-containing salts.

Chelating agents can be added to the medium in order to keep the metal ions in solution. Particularly suitable chelating agents comprise dihydroxyphenols, such as catechol or protocatechuate, or organic acids, such as citric acid.

The fermentation media employed according to the invention customarily also contain other growth factors, such as vitamins or growth promoters, which include, for example, biotin, riboflavin, thiamine, folic acid, nicotinic acid, pantothenate and pyridoxine. Growth factors and salts are often derived from complex media components, such as yeast extract, molasses, corn-steep liquor and the like. Suitable precursors can moreover be added to the culture medium. The exact composition of the media compounds depends strongly on the particular experiment and is decided individually for each specific case. Information about media optimization is obtainable from the textbook “Applied Microbiol. Physiology, A Practical Approach” (Ed. P. M. Rhodes, P. F. Stanbury, IRL Press (1997) pp. 53-73, ISBN 0 19 963577 3). Growth media can also be obtained from commercial suppliers, such as Standard 1 (Merck) or BHI (Brain heart infusion, DIFCO) and the like.

All media components are sterilized, either by heat (20 min at 1.5 bar and 121° C.) or by sterile filtration. The components can either be sterilized together or if necessary separately. All media components can be present at the start of growth or can optionally be added continuously or batchwise.

The temperature of the culture is normally between 15° C. and 45° C., preferably 25° C. to 40° C., and can be kept constant or changed during the experiment. The pH of the medium should be in the range from 5 to 8.5, preferably around 7.0. The pH for the propagation can be controlled during the propagation by addition of basic compounds such as sodium hydroxide, potassium hydroxide, ammonia or ammonia water or acidic compounds such as phosphoric acid or sulfuric acid. For the control of foam development, it is possible to employ antifoams such as, for example, fatty acid polyglycol esters. For the maintenance of the stability of plasmids, suitable selectively acting substances, such as, for example, antibiotics, can be added to the medium. In order to maintain aerobic conditions, oxygen or oxygen-containing gas mixtures, such as, for example, ambient air, are introduced into the culture. The temperature of the culture is normally 20° C. to 45° C. and. The culture is continued until a maximum of the desired product has formed. This aim is normally achieved within 10 hours to 160 hours.

The fermentation broth is subsequently processed further. According to demand, the biomass can be removed completely or partially from the fermentation broth by separation methods, such as, for example, centrifugation, filtration, decanting or a combination of these methods, or left in it completely.

The cells can also be disrupted, if the polypeptides are not secreted into the culture medium, and the product recovered from the lysate according to known protein isolation processes. The cells can alternatively be disrupted by high-frequency ultrasound, by high pressure, such as, for example, in a French pressure cell, by osmolysis, by action of detergents, lytic enzymes or organic solvents, by homogenizers or by combination of several of the processes mentioned.

A purification of the polypeptides can be achieved using known, chromatographic processes, such as molecular sieve chromatography (gel filtration), such as Q-Sepharose chromatography, ion-exchange chromatography and hydrophobic chromatography, and using other customary processes such as ultrafiltration, crystallization, salting out, dialysis and native gel electrophoresis. Suitable processes are described, for example, in Cooper, F. G., Biochemische Arbeitsmethoden [Biochemical Working Methods], Verlag Walter de Gruyter, Berlin, New York or in Scopes, R., Protein Purification, Springer Verlag, New York, Heidelberg, Berlin.

It can be advantageous for the isolation of the recombinant protein to use vector systems or oligonucleotides that elongate the cDNA by specific nucleotide sequences and thus code for modified polypeptides or fusion proteins that serve, for example, for simpler purification. Suitable modifications of this type are, for example, “tags” functioning as anchors, such as, for example, the modification known as a hexa-histidine anchor or epitopes that can be recognized as antigens by antibodies (described, for example, in Harlow, E. and Lane, D., 1988, Antibodies: A Laboratory Manual. Cold Spring Harbor (N.Y.) Press). These anchors can serve for the attachment of the proteins to a solid carrier, such as, for example, a polymer matrix, that can be filled, for example, into a chromatography column, or can be used on a microtiter plate or on some other carrier.

At the same time, these anchors can also be used for the recognition of the proteins. For recognition of the proteins, customary markers, such as fluorescent dyes, enzyme markers that after reaction with a substrate form a detectable reaction product, or radioactive markers, can moreover be used alone or in combination with the anchors for derivatization of the proteins.

### 3.6 Enzyme Immobilization

The enzymes according to the invention can be employed in free or immobilized form in the processes described herein. An immobilized enzyme is understood as meaning an enzyme that is fixed to an inert carrier. Suitable carrier materials and the enzymes immobilized thereon are known from EP-A-1149849, EP-A-1 069 183 and DE-A 100193773 and from the references cited therein. Reference is made fully in this regard to the disclosure of these specifications. The suitable carrier materials include, for example, clays, clay minerals, such as kaolinite, diatomaceous earths, perlite, silica, alumina, sodium carbonate, calcium carbonate, cellulose powder, anion exchange materials, synthetic polymers, such as polystyrene, acrylic resins, phenol-formaldehyde resins, polyurethanes and polyolefins, such as polyethylene and polypropylene. The carrier materials are customarily employed in a finely divided, particulate form for the production of the supported enzymes, porous forms being preferred. The particle size of the carrier material is customarily not more than 5 mm, in particular not more than 2 mm (grading curve). Analogously, on use of the dehydrogenase as a whole-cell catalyst a free or immobilized form can be chosen. Carrier materials are, for example, Ca alginate and carrageenan. Enzymes as well as cells can also be crosslinked directly using glutaraldehyde (crosslinking to CLEAs). Corresponding and further immobilization processes are described, for example, in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim.

### Experimental Section

If no other information is given, the cloning steps carried out in the context of the present invention, such as, for example, restriction cleavages, agarose gel electrophoresis, purification of DNA fragments, transfer of nucleic acids to nitrocellulose and nylon membranes, linkage of DNA fragments, transformation of microorganisms, propagation of microorganisms, replication of phages and sequence analysis of recombinant DNA were can be carried out as described in Sambrook et al. (1989) loc. cit.

### A. General Information

### Materials:

Enzymes and enzyme buffers were obtained from Fermentas, St. Leon-Rot or NEB, Frankfurt.

### LB Medium:

| | | |
|---|---|---|
| Bacto tryptone | 10 | g |
| yeast extract | 5 | g |
| sodium chloride | 5 | g |
| double-distilled water | to 1000 | ml |

### TB Medium:

### Solution I:

| Bacto tryptone | 12 | g |
|---|---|---|
| yeast extract | 24 | |
| glycerol, anhydrous | 4 | ml |
| double-distilled water | to 900 | ml |

### Solution II:

| | |
|---|---|
| potassium dihydrogenphosphate | 0.17M |
| potassium hydrogen phosphate | 0.72M |
| double-distilled water | to 100 ml |

The two solutions were combined after autoclaving.

### Expression Vectors

For the expression of 12α-HSDH, the vector pET22b(+), Novagen, Darmstadt was used, which contains an MCS under the control of a T7 promoter and transcription start and a T7 terminator. The expression is induced by means of isopropyl β-D-thiogalactopyranoside (IPTG).

To this end, 12α-HSDH-encoding sequences were PCR-amplified. The PCR products were obtained using the genomic DNA of Clostridium sp. group P strain 48-50 as a template and the primer pair described more precisely later. The PCR products were applied to an agarose gel, separated and excized from this. Subsequently, they were restricted with the aid of NdeI and BamHI and ligated with the pET22b(+) vector likewise cleaved with NdeI and BamHI.

### Microorganisms

| Strain | Genotype |
|---|---|
| Clostridium sp. group P strain 48-50 | |
| Escherichia coli BL21 (DE3) | F⁻ompT gal dcm Ion |
| | hsdS_{B}(r_{B}⁻m_{B}⁻)λ(DE3 |
| | [lacI lacUV5-T7 gene |
| | 1 ind1 sam7 nin5]) |
| Escherichia coli Rosetta ™ (DE3) | F⁻ompT hsdS_{B}(R_{B}⁻m_{B}⁻) |
| | gal dcm λ(DE3 [lacI |
| | lacUV5-T7 gene 1 |
| | ind1 sam7 nin5]) |
| | pLysS-RARE(Cam^{R}) |

### Methods

### 1. Standard Conditions for 12α-HSDH Activity Determination

The activity is defined as follows: 1 U of the enzyme corresponds to the amount of enzyme which catalyzes the reaction of 1 μmol/min of a 5 mM cholic acid solution in potassium phosphate buffer (50 mM, pH 8.0) at room temperature (i.e. about 20° C.-23° C.).

For the activity determination, 790 μl of potassium phosphate buffer (50 mM, pH 8.0), 100 μm of cholic acid (50 mM in potassium phosphate buffer (50 mM, pH 8.0)) and 10 μl of enzyme solution to be measured were mixed in a cuvette. 100 μl of NADP⁺ (2.5 mM) was added at the start of the reaction and the increase in the absorption at 340 nm was determined photometrically. The gradient over 30 s was determined at RT. Determination of the activity took place according to the Lambert-Beer's law.

### 2. Protein Concentration Determination by Means of BCA Assay

The protein concentration of a solution was determined by measuring the absorption of 20 μl of protein solution, such as, for example, of a cell lysate dilution or of a resuspended cell debris pellet after ultrasonic disruption, in 200 μl of BOA solution (solution A:solution B 50:1) of the analysis kit of Bio-Rad, Munich at 562 nm. Here, the bicinchoninic acid (BCA) forms, with monovalent copper ions that result quantitatively from the reduction of bivalent copper ions by the protein, a violet complex compound whose absorption at 562 nm can be measured photometrically. The determination of the concentration was carried out by means of a bovine serum albumin (BSA) calibration line.

### B. Preliminary Experiments for Gene Isolation

It is the aim of all experimental studies to find an improved access to the enzyme 12α-HSDH. In order to achieve this aim, the sequence of the gene coding for 12α-HSDH was elucidated according to the invention.

1.1 Firstly, this was attempted by polymerase chain reaction (PCR) using degenerate oligonucleotide primers. The oligonucleotides employed were on the one hand constructed based on the published N-terminal amino acid sequence (cf. Braun et al., loc. cit.) of 12α-HSDH. In order to keep the degree of degeneration low, the primers were derived beginning with the N-terminal methionine (only one codon) (primer sequences not shown). On the other hand, databank-supported sequence comparisons showed that a conserved amino acid motif “LVNN” is present in HSDHs. This region was used for the construction of a reverse primer. Additionally, a further, less strongly conserved sequence motif PE(Q)DIAN was used for the design of degenerate primers (primer sequences not shown). Further degenerate oligonucleotides were discarded by means of the freely accessible programme CODEHOP (Rose, T. M. et al., Nucleic Acids Research, 1998, 26(7), 1628-1635).

It was not possible to amplify the gene sought using all combinations of the degenerate primer pairs indicated above.

1.2 In an experiment for the determination of further peptide sequence fragments of 12α-HSDH, it was attempted to purify the enzyme from the lysate of Clostridium sp. group P by means of a combination of two affinity chromatography steps. This process was described by Braun et al, loc. cit., but could not be reworked.

### C. Isolation of the Encoding 12α-HSDH Sequence and Characterization of the 12α-HSDH Enzyme

### Example 1

### Sequence Homology Investigations

In order to obtain access to the 12α-HSDH sequence, the genome of Clostridium sp. group P strain 48-50 DSM 4029 was sequenced. The search both for the published N-terminal sequence and the search for the motif “LYNN” in all open reading frames (ORE's) did not lead to the sequence of the gene coding for 12α-HSDH.

It was possible only by sequence homology comparisons to identify a gene that contained the published partial sequence information in a modified form. The comparisons were carried out with TBLASTX (Tatusova and Madden (1999) FEMS Microbiol Lett 174 (2), 247-250) and under the following conditions:

### Open gap: 5

### Extension gap: 2

gap x_dropoff: 50
expect: 10.0
word size: 11

The standard conditions of http://www.ncbi.nlm.nih.gov/blast/Blast.cgi?PAGE=Translations&PROGRAM=tblastx&BLAST PROGRAMS=tblastx&PAGE TYPE-Blast Search&SHOW DEFAULTS=on#i were used; parameters are to be found there under “Algorithm parameters”.

In the gene found (SEQ ID NO: 3), the N-terminal methionine indicated in the published partial sequence is missing; moreover, the published partial sequence is not to be found at the N-terminus of the protein (bio-informatic prediction of the gene start with GLIMMER, CBCB, Maryland, USA). The conserved motif “LVNN” is also modified in 12α-HSDH to “LINN” (SEQ ID NO: 5).

For these reasons, it was not possible to successfully run the originally followed approach for sequence elucidation using degenerate oligonucleotides. Methionine in particular is regularly used in order to derive degenerate primers, as it is only encoded by a single base triplet. In the same way, it was not to be expected that calculated 12α-HSDH from Clostridium sp. shows deviations in the conserved sequence “LVNN”.

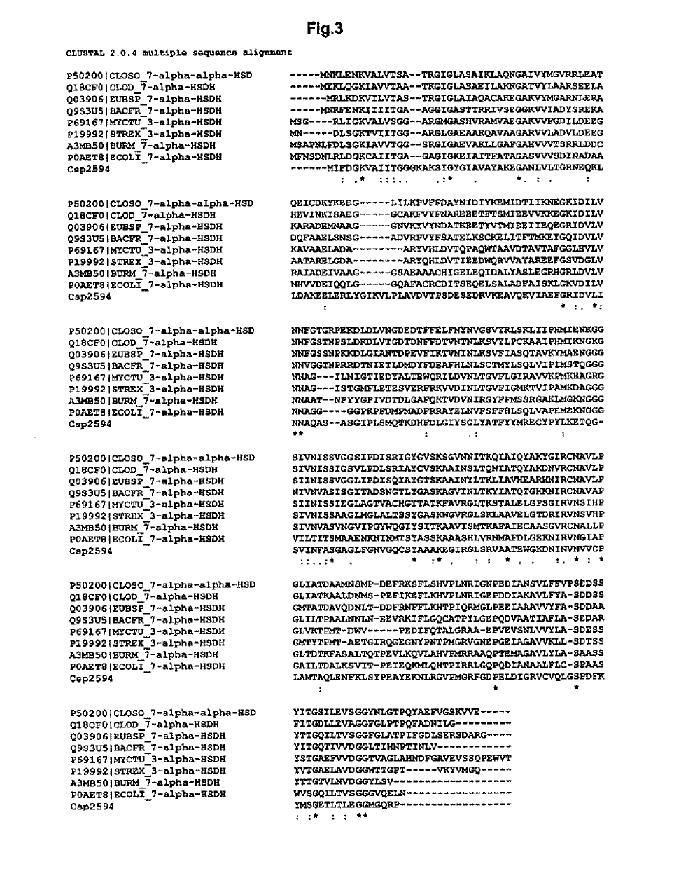

FIG. 3 shows a partial multi-sequence alignment between known microbial HSDH and HSDH according to the invention.

### Example 2

### Amplification of the 12α-HSDH Gene and Expression of 12α-HSDH

### 1. Amplification

The following primers were used for this:

Forward long (long enzyme version,
NdeI cleavage site):
(SEQ ID NO: 14)
GGTATTCCATATGGATTTTATTGATTTTAAGGAGATG.
Forward short (short enzyme version,
NdeI cleavage site):
(SEQ ID NO: 15)
GGTATTCCATATGATCTTTGACGGAAAGGTCGC.
Primer reverse (BamH1 cleavage site)
(SEQ ID NO: 16)
CGGGATCCCTAGGGGCGCTGACCC.

The target gene was amplified by PCR using Pfu polymerase.

As a template, the genomic DNA of Clostridium sp. group P strain 48-50 DSM 4029 (29.4 ng/μl) was used, of which 1 μl was employed. For amplification, 1 μl of the Pfu polymerase was used. The buffer used was Pfu buffer (10× with MgSO₄) (Fermentas, St. Leon-Rot). In each case 1.5 μl of forward and reverse primer (10 μM) were employed, and 2 μl of deoxynucleotide triphosphate (20 μM). The batch was adjusted to 50 μl with RNase-free water. The reaction was carried out in the Eppendorf thermocycler. The PCR batch was initially started at 95° C. for 5 min in order to denature the DNA. Then, in the cloning of the unknown DNA sequences, 30 cycles followed beginning with a denaturation at 95° C. for 30 s. Subsequently, the batch was cooled to 25-45° C. in each case for 30 s by means of a temperature gradient in order to guarantee annealing of the degenerate primer on the target DNA (constant annealing temperature of 53° C.). Thereupon, a temperature of 72° C. for 90 s was adjusted for primer extension since the activity optimum of the polymerase used lies here. Finally, the batches were incubated at 72° C. for 10 min and cooled at 4° C. until removal from the apparatus.

### 2. Expression

After amplification by means of polymerase chain reaction, the target gene was cloned into the expression vector pET22b+ by means of the cleavage sites NdeI and BamHI, introduced into E. coli Rosetta DE3™ and E. coli BL21 DE3 cells and expressed. These are nonpathogenic strains that make possible the production of large amounts of the enzyme (up to 150 000 U/l of culture).

For expression, 5 ml of LB medium (with 100 μg/ml of ampicillin) were incubated at 37° C. and 180 rpm with the E. coli BL21 (DE) or Rosetta™ clone, which was transformed with an expression vector, for 16 h. 200 ml of TB medium (with 100 μg/ml of ampicillin) was inoculated therewith and incubated at 37° C. and 180 rpm. The expression of 12α-HSDH was induced with 1 mM IPTG at an OD₆₀₀ of 0.6-0.8. At various times, 1 ml of cell suspension with an OD₆₀₀ of 0.25 was removed, pelleted for 1 min at 13 000 rpm and stored at −20° C. until further use. The expression was ended after 4 or 22 h by pelleting the cells at 2700 g for 10 min at 4° C. and subsequently freezing them.

The cells were disrupted with the aid of ultrasound by firstly resuspending the pellets in 4-10 ml of potassium phosphate buffer (50 mM, pH 8.0). The cells were treated in an ice bath in an ultrasonic disintegrator from Branson at an intensity of 30% four times for 1 min with a 2 min break in each case. Subsequently, the cell debris was removed by centrifugation for 1 h at 4220 g and 4° C.

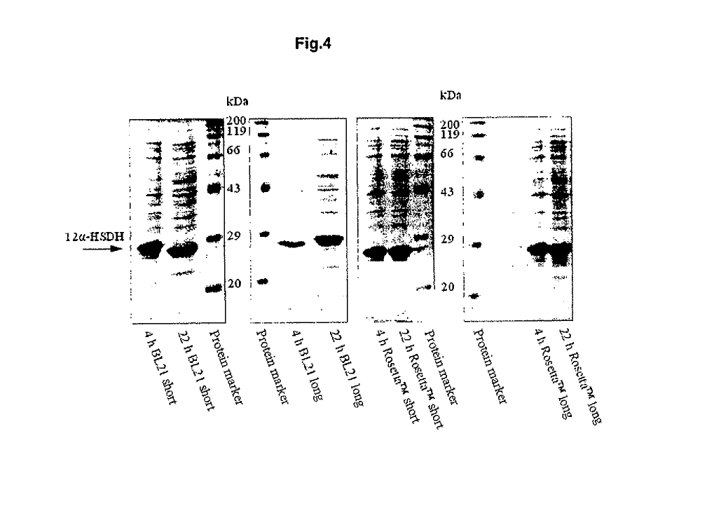

FIG. 4 shows the results of an SDS-PAGE (12.5% strength gel, β-mercaptoethanol) of cell lysates after 4 h and 22 h expression of enzymes according to the invention (HSDH_short and HSDH_long) (band in each case at approximately 27 kDa).

Protein contents and enzyme activities were determined as described above.

The enzyme activities achieved after disruption of the E. coli cells are summarized in the following table.

| | Expres- | Activity [of culture medium] | | |
|---|---|---|---|---|
| Strain | sion | | | pET22b-empty |
| (E. coli) | period [h] | HSDH_long | HSDH_short | vector |
| Rosetta ™ | 4 | 3100 | 3400 | 200 |
| (DE3) | 22 | 19500 | 24300 | 0 |
| BL21 (DE3) | 4 | 12700 | 12800 | — |
| | 22 | 36800 | 33000 | — |

Owing to the high expression level, the volumetric and specific activity of the enzyme preparation after cell disruption and centrifugation is already markedly higher (36 800 U/l of culture medium) than that originating from Clostridium sp. group P strain 48-50. Thus in contrast to the previously described processes, the protein purification can be dispensed with.

The high proportion of the target protein in the total amount of protein of BL21 (DE3) cells is illustrated by the following table:

| | Expres- | Activity [of culture medium] | | |
|---|---|---|---|---|
| | sion | | | pET22b-empty |
| Strain | period [h] | HSDH_long | HSDH_short | vector |
| Rosetta ™ | 4 | 3100 | 3400 | 200 |
| (DE3) | 22 | 19500 | 24300 | 0 |
| BL21 (DE3) | 4 | 12700 | 12800 | — |
| | 22 | 36800 | 33000 | — |

### Example 3

### Preparative Synthesis of 12-Ketochenodeoxy-Cholic Acid from Cholic Acid

The expressed enzyme (short version) was employed in combination with ADH t (Codexis, Jülich) for the preparative synthesis of 12-ketochenodeoxycholic acid. For this, 500 ml of cholic acid (400 mM in potassium phosphate buffer (50 mM, pH 8.0), 10% acetone), 0.25 mM NADP⁺, 2000 U of 12α-HSDH_short from E. coli BL21 (DE 3) (cf. above Example 2) and, for cofactor regeneration, 550 U of ADH t (from Thermoanaerobacter sp., Codexis, Jülich were mixed in a 1 l round-bottomed flask. The reaction was carried out at RT, with continuous stirring and reflux cooling. After 27 h, a further 550 U of 12α-HSDH and 138 U of ADH t were added and the mixture was incubated for a total of 117 h. During the reaction, the photometric absorption was determined at 340 nm and 1 ml samples were removed for monitoring the course of the reaction, which were stopped with 100 μl of hydrochloric acid (1 M) and evaporated or extracted in ethyl acetate. The absorption course is shown in FIG. 5.

The reaction was acidified by addition of fuming hydrochloric acid (37%) until complete precipitation of the reaction partners. The supernatant was removed and extracted three times with 50 ml of ethyl acetate in each case. The precipitated cholic acid derivatives were completely dissolved in acetone with addition of hydrochloric acid and warming. The organic phases were combined and dried until free of solvent.

It turned out here that the product extraction is markedly simpler to bring about than with the use of the commercial product directly from Clostridium sp. Group P strain 48-50 (ASA Spezialenzyme). The reason for this is the markedly lower total protein content with identical HSDH activity.

### Example 4

### Characterization of the Expressed Enzymes

The expressed enzymes were characterized with respect to their activity. It appeared that the selective oxidation of cholic acid to 12-ketochenodeoxycholic acid is catalyzed.

The reference substances cholic acid and 12-ketocheno-deoxycholic acid and extracts of the reaction batches (Example 3) were applied to a TLC silica gel 60 F₂₅₄ aluminum foil, Merck, Darmstadt by means of a glass capillary. The foil was placed as vertically as possible in a chromatography chamber that contained as eluent a mixture of dichloromethane:acetone:acetic acid (conc.) in the ratio 40:40:3. The separation was carried out until the eluent front had almost reached the upper edge of the plate. The coloration of the substances was carried out by means of spraying with molybdatophosphoric acid spray reagent (40 mM molybdatophosphoric acid, 95.2% conc. acetic acid, 4.8% conc. sulfuric acid) and subsequent heating.

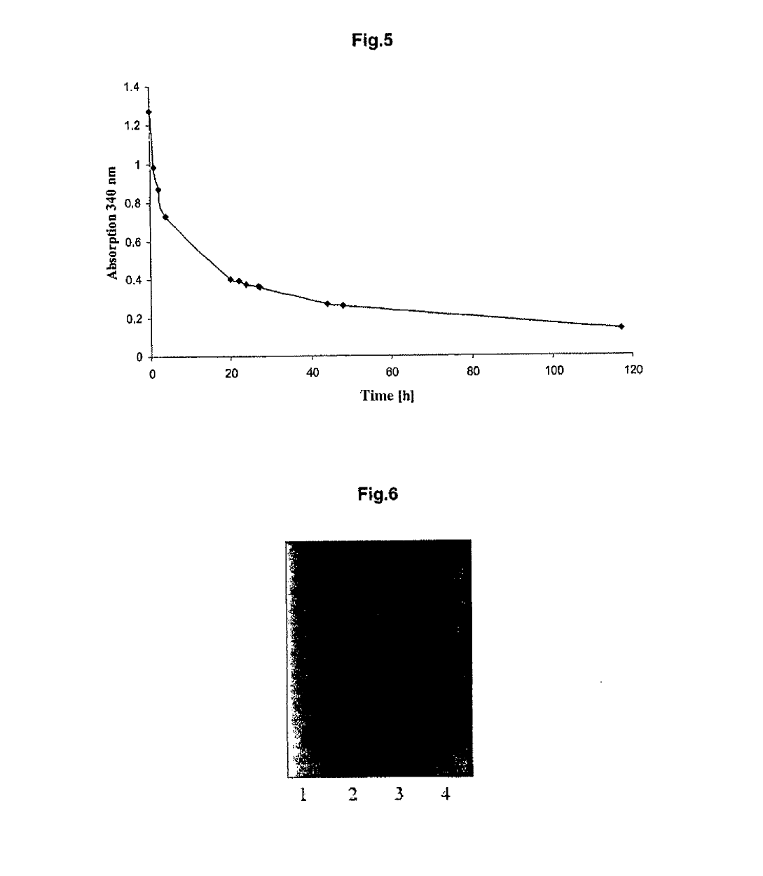

The results are shown in FIG. 6.

### Example 5

### Location of Amino Acid Residues Involved in NADPH Binding

By means of homology comparisons with NADH and NADPH-dependent “short chain dehydrogenases” (SDR), it was possible to identify two amino acid side chains important for cofactor recognition and possibly for future cofactor discrimination: by site-directed mutagenesis (substitutions were prepared based on publications (Tanaka et al. (1996) Biochemistry 35(24): 7715-30) and (Carugo and Argos (1997) Proteins 28(1): 10-28)), the substitutions G37D and R38L (based on SEQ ID NO: 3) were carried out. The experiments were carried out according to the experimental details for the QuikChange Site-directed Mutagenesis Kit of Stratagene GmbH.

The primers (see following table) for the site-directed mutagenesis were chosen on the basis of the 12α-HSDH gene sequence such that they brought about the desired amino acid exchange. Care was taken here that the mutation (marked underlined) was located centrally in the primer and the melt temperature of two primer pairs was situated in the same range. The following combinations were used:

MBr_QC_HSDH_G37D_forw/MBr_QC_HSDH_G37D_rev with pET22b(+)-HSDH_short and
MBr_QC_HSDH_R38L_forw/MBr_QC_HSDH_R38L_rev with pET22b(+)-HSDH_short_G37D.

### Primers for Position-Directed Mutagenesis

| | | Melt |
|---|---|---|
| Primer | Sequence | temperature |
| MBr_QC_ | 5′-CTGGTCCTGACCG | 63° C. |
| HSDH_G37D_ | ACAGAAACGAGC-3′ | |
| forw | (SEO ID NO: 17) | |
| MBr_QC_ | 5′-GCTCGTTTCTGTC | |
| HSDH_G37D_ | GGTCAGGACCAG-3′ | 63° C. |
| rev | (SEQ ID NO: 18) | |
| MBr_QC_ | 5′-GTCCTGACCGACTT | |
| HSDH_R38L_ | AAACGAGCAGAAAC-3′ | 61° C. |
| forw | (SEO ID NO: 19) | |
| MBr_QC_ | 5′-GTTTCTGCTCGTTTAA | |
| HSDH_R38L_ | GTCGGTCAGGAC-3′ | 61° C. |
| rev | (SEO ID NO: 20) | |

It turned out that the resulting protein variants no longer showed activity with NADPH. This underlines the importance of the identified positions for the cofactor binding. The variants thus prepared hitherto showed no activity with NADH. However, an NADH-dependent HSDH variant could be obtained by saturation mutagenesis at the positions described or further positions.

### Example 6

### Characterization of the Product Inhibition Mutant 37D12

In the 12α-HSDH investigated, inhibition by the product 12-ketochenodeoxycholic acid is to be observed, which can have a negative effect on the reaction rate in the process. In order to reduce this product inhibition of the 12α-HSDH, a random-based 12α-HSDH library with 4000 mutants was prepared by means of error-prone PCR. Suitable methods are known in principle and described, for example, in: Cadwell, R. C. et al., Randomization of Genes by PCR Mutagenesis; (1992) PCR Methods and Applications, 2:28-33, Cold Spring Harbor Laboratory; Arnold, F. H. et al., Current Opinion in Chemical Biology (1999) 3:54-59; or Liebeton, K. et al., Chemistry & Biology, (2000), 7:709-718. The starting amount of the target DNA for the error-prone PCR was chosen such that a mutation rate of 4.5 mutations per kb was achieved. The product was ligated into the pET22b(+) vector and transformed in E. coli Nova Blue (DE3).

A number of approximately 4000 mutants were picked in microtiter plates (MTP), which served for the inoculation of the main cultures. For induction, expression and cell disruption, MTP with deep cavities were used. The screening of the cell lysates of all 4000 mutants was carried out on the microtiter scale in the presence of product. Several mutants were identified here, the mutant 37D12 being used further.

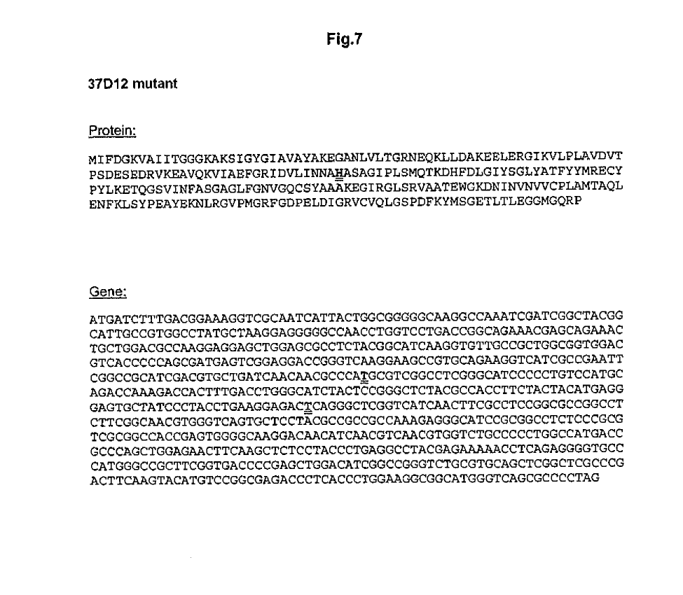

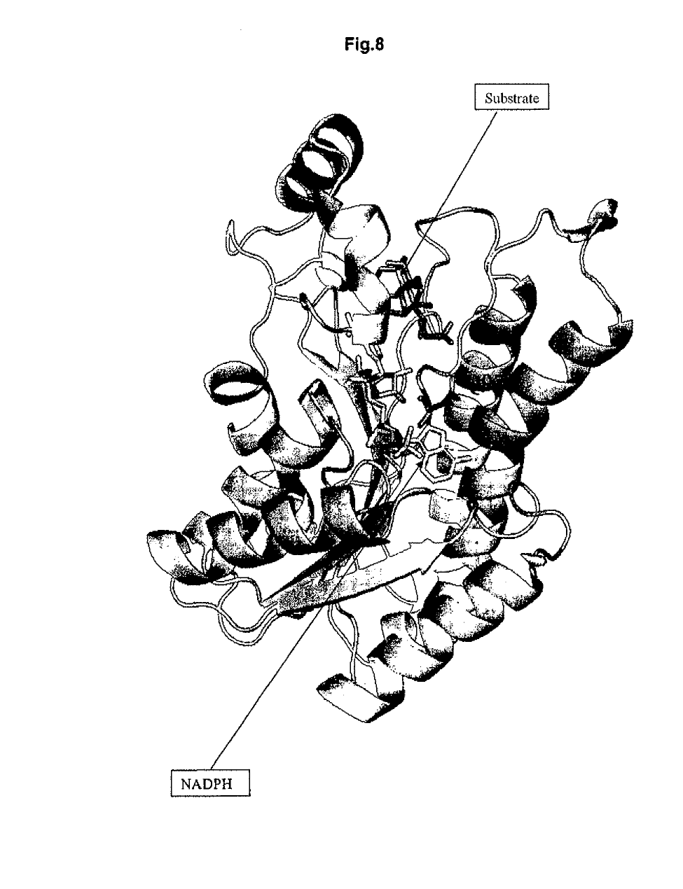

The mutation of the mutant 37D12 in the 12α-HSDH homology model can be seen in FIG. 8. It is an exchange of glutamine for histidine (cf. Sequences according to FIG. 7) and is located in the region of the active center between the substrate and cofactor binding pocket.

Since the mutant 37D12 had modified kinetics compared to the wild-type, for the further analysis of the product inhibition the activity was defined such that the time range of 30 sec within the first minute after the start of the reaction, in which the highest linearity was achieved, was employed for the calculation of the activity. After the wild-type and the mutant 37D12 were purified by means of metal affinity chromatography, the product inhibition was investigated again using these conditions. As illustrated in FIG. 9, the mutant shows a markedly reduced inhibition even at a turnover of 1%. At 5% turnover, the wild-type enzyme showed a loss of 60% in contrast to 20% for the mutant 37D12. The three-fold activity remained in the case of the mutant 37012 compared to the wild-type at a turnover of 25%.

After the purification, it was possible to calculate the specific activity of the mutant to be 15.71 U/mg and of the wild-type to be 30.87 U/mg. The mutation therefore results in an activity loss of about 50%.

Assignment of the SEQ ID NOs:

| SEQ ID NO: | Description | Type |
|---|---|---|
| 1 | 12α-HSDH; L | NS |
| 2 | 12α-HSDH; L | AS |
| 3 | 12α-HSDH; S | NS |
| 4 | 12α-HSDH; S | AS |
| 5 | 12α-HSDH sequence motif; L and S | AS |
| 6 | N-terminus; L | AS |
| 7 | N-terminus; L | AS |
| 8 | Sequence motif; L and S | AS |
| 9 | N-terminal sequence motif; S | AS |
| 10 | N-terminal sequence motif; S | AS |
| 11 | N-terminal sequence motif; L | AS |
| 12 | Sequence motif; L and S | AS |
| 13 | C-terminal sequence motif; L and S | AS |
| 14 | PCR primer; L | NS |
| 15 | PCR primer; S | NS |
| 16 | PCR primer; L and S | NS |
| 17 | PCR primer | NS |
| 18 | PCR primer | NS |
| 19 | PCR primer | NS |
| 20 | PCR primer | NS |
| 21 | Mutant 37D12; S | NS |
| 22 | Mutant 37D12; S | AS |

AS = amino acid sequence
NS = nucleic acid sequence
L = long version
S = short version

Reference is expressly made here to the disclosure of the publications cited herein.

1-28. (canceled)

29. A process for the preparation of ursodeoxycholic acid (UDCA) of the formula (1) 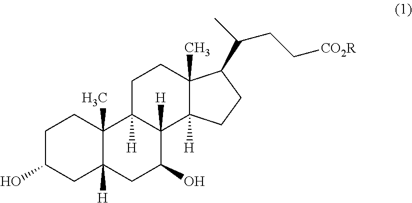
in which
R represents alkyl, NR¹R², H, an alkali metal ion or N(R³)₄⁺, in which the radicals R³ are identical or different and represent H or alkyl, where
a) a cholic acid (CA) of the formula (2) 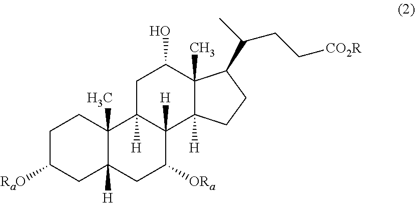
in which R has the meanings indicated above, and the radicals Rₐ are identical or different and represent H or acyl, is oxidized in the presence of a 12α-hydroxysteroid dehydrogenase, wherein the 12α-hydroxysteroid dehydrogenase is recombinantly produced by expression of a polynucleotide consisting of SEQ ID NO:3 in a non-pathogenic microorganism, to the corresponding 12-ketochenodeoxycholic acid (12-keto-CDCA) of the formula (3) 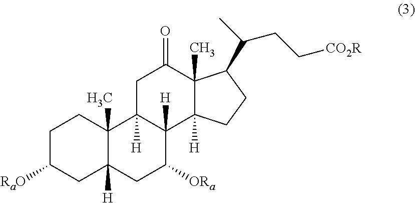
in which R and Rₐ have the meanings indicated above, and subsequently
b) 12-keto-CDCA of the formula (3) is reacted by deoxygenation to give chenodeoxycholic acid (CDCA) of the formula (4) 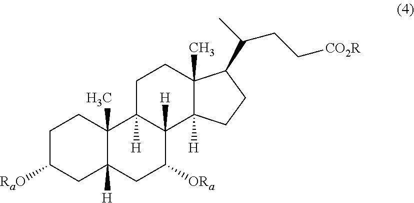
in which R and Rₐ have the meanings indicated above, and
c) CDCA of the formula (4) is chemically oxidized in position 7 to the 7-ketolithocholicacid (KLCA) of the formula (5) 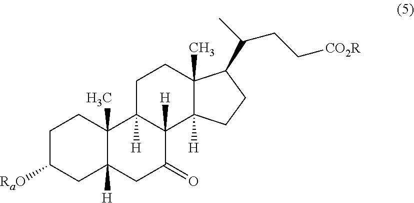
in which R and Rₐ have the meanings indicated above; and
d) KLCA of the formula (5) is reduced and
e) the reaction product is optionally further purified.

30. The process as claimed in claim 29, where, if Ra represents acyl, this acyl group is optionally removed after carrying out the reaction step b) or d).

31. The process as claimed in claim 29, where step a) takes place in the presence of nicotinamide adenine dinucleotide phosphate NAD(P)⁺.

32. The process as claimed in claim 31, where NAD(P)⁺ consumed are regenerated electrochemically or enzymatically.

33. The process as claimed in claim 29, where step a) takes place with a 12α-hydroxysteroid dehydrogenase in immobilized form.


SEQUENCE LISTING (22 sequences)

SEQ ID NO: 1 - DNA, 813 bp, Clostridium sp.
  CDS (1)..(810)
atggattttattgattttaaggagatgggcagaatggggatctttgacggaaaggtcgcaatcattactggcgggggcaaggccaaatcgatcggctacggcattgccgtggcctatgctaaggagggggccaacctggtcctgaccggcagaaacgagcagaaactgctggacgccaaggaggagctggagcgcctctacggcatcaaggtgttgccgctggcggtggacgtcacccccagcgatgagtcggaggaccgggtcaaggaagccgtgcagaaggtcatcgccgaattcggccgcatcgacgtgctgatcaacaacgcccaggcgtcggcctcgggcatccccctgtccatgcagaccaaagaccactttgacctgggcatctactccgggctctacgccaccttctactacatgagggagtgctatccctacctgaaggagacccagggctcggtcatcaacttcgcctccggcgccggcctcttcggcaacgtgggtcagtgctcctacgccgccgccaaagagggcatccgcggcctctcccgcgtcgcggccaccgagtggggcaaggacaacatcaacgtcaacgtggtctgccccctggccatgaccgcccagctggagaacttcaagctctcctaccctgaggcctacgagaaaaacctcagaggggtgcccatgggccgcttcggtgaccccgagctggacatcggccgggtctgcgtgcagctcggctcgcccgacttcaagtacatgtccggcgagaccctcaccctggaaggcggcatgggtcagcgcccctag

SEQ ID NO: 2 - protein, 270 aa, Clostridium sp.
MDFIDFKEMGRMGIFDGKVAIITGGGKAKSIGYGIAVAYAKEGANLVLTGRNEQKLLDAKEELERLYGIKVLPLAVDVTPSDESEDRVKEAVQKVIAEFGRIDVLINNAQASASGIPLSMQTKDHFDLGIYSGLYATFYYMRECYPYLKETQGSVINFASGAGLFGNVGQCSYAAAKEGIRGLSRVAATEWGKDNINVNVVCPLAMTAQLENFKLSYPEAYEKNLRGVPMGRFGDPELDIGRVCVQLGSPDFKYMSGETLTLEGGMGQRP

SEQ ID NO: 3 - DNA, 774 bp, Artificial Sequence
  Synthetic polynucleotide
atctttgacggaaaggtcgcaatcattactggcgggggcaaggccaaatcgatcggctacggcattgccgtggcctatgctaaggagggggccaacctggtcctgaccggcagaaacgagcagaaactgctggacgccaaggaggagctggagcgcctctacggcatcaaggtgttgccgctggcggtggacgtcacccccagcgatgagtcggaggaccgggtcaaggaagccgtgcagaaggtcatcgccgaattcggccgcatcgacgtgctgatcaacaacgcccaggcgtcggcctcgggcatccccctgtccatgcagaccaaagaccactttgacctgggcatctactccgggctctacgccaccttctactacatgagggagtgctatccctacctgaaggagacccagggctcggtcatcaacttcgcctccggcgccggcctcttcggcaacgtgggtcagtgctcctacgccgccgccaaagagggcatccgcggcctctcccgcgtcgcggccaccgagtggggcaaggacaacatcaacgtcaacgtggtctgccccctggccatgaccgcccagctggagaacttcaagctctcctaccctgaggcctacgagaaaaacctcagaggggtgcccatgggccgcttcggtgaccccgagctggacatcggccgggtctgcgtgcagctcggctcgcccgacttcaagtacatgtccggcgagaccctcaccctggaaggcggcatgggtcagcgcccctag

SEQ ID NO: 4 - protein, 257 aa, Artificial Sequence
  Synthetic polypeptide
IFDGKVAIITGGGKAKSIGYGIAVAYAKEGANLVLTGRNEQKLLDAKEELERLYGIKVLPLAVDVTPSDESEDRVKEAVQKVIAEFGRIDVLINNAQASASGIPLSMQTKDHFDLGIYSGLYATFYYMRECYPYLKETQGSVINFASGAGLFGNVGQCSYAAAKEGIRGLSRVAATEWGKDNINVNVVCPLAMTAQLENFKLSYPEAYEKNLRGVPMGRFGDPELDIGRVCVQLGSPDFKYMSGETLTLEGGMGQRP

SEQ ID NO: 5 - protein, 4 aa, Artificial Sequence
  Synthetic peptide
LINN

SEQ ID NO: 6 - protein, 41 aa, Artificial Sequence
  Synthetic polypeptide
MDFIDFKEMGRMGIFDGKVAIITGGGKAKSIGYGIAVAYAK

SEQ ID NO: 7 - protein, 14 aa, Artificial Sequence
  Synthetic peptide
MDFIDFKEMGRMGI

SEQ ID NO: 8 - protein, 15 aa, Artificial Sequence
  Synthetic peptide
ITGGGKAKSIGYGIA

SEQ ID NO: 9 - protein, 5 aa, Artificial Sequence
  Synthetic peptide
IFDGK

SEQ ID NO: 10 - protein, 6 aa, Artificial Sequence
  Synthetic peptide
GIFDGK

SEQ ID NO: 11 - protein, 6 aa, Artificial Sequence
  Synthetic peptide
RMGIFD

SEQ ID NO: 12 - protein, 7 aa, Artificial Sequence
  Description of Artificial Sequence Synthetic peptide
VLTGRNE

SEQ ID NO: 13 - protein, 8 aa, Artificial Sequence
  Synthetic peptide
FGDPELDI

SEQ ID NO: 14 - DNA, 37 bp, Artificial Sequence
  Synthetic primer
ggtattccatatggattttattgattttaaggagatg

SEQ ID NO: 15 - DNA, 33 bp, Artificial Sequence
  Synthetic primer
ggtattccatatgatctttgacggaaaggtcgc

SEQ ID NO: 16 - DNA, 24 bp, Artificial Sequence
  Synthetic primer
cgggatccctaggggcgctgaccc

SEQ ID NO: 17 - DNA, 25 bp, Artificial Sequence
  Synthetic primer
ctggtcctgaccgacagaaacgagc

SEQ ID NO: 18 - DNA, 25 bp, Artificial Sequence
  Synthetic primer
gctcgtttctgtcggtcaggaccag

SEQ ID NO: 19 - DNA, 28 bp, Artificial Sequence
  Synthetic primer
gtcctgaccgacttaaacgagcagaaac

SEQ ID NO: 20 - DNA, 28 bp, Artificial Sequence
  Synthetic primer
gtttctgctcgtttaagtcggtcaggac

SEQ ID NO: 21 - DNA, 777 bp, Artificial Sequence
  Synthetic polynucleotide
atgatctttgacggaaaggtcgcaatcattactggcgggggcaaggccaaatcgatcggctacggcattgccgtggcctatgctaaggagggggccaacctggtcctgaccggcagaaacgagcagaaactgctggacgccaaggaggagctggagcgcctctacggcatcaaggtgttgccgctggcggtggacgtcacccccagcgatgagtcggaggaccgggtcaaggaagccgtgcagaaggtcatcgccgaattcggccgcatcgacgtgctgatcaacaacgcccatgcgtcggcctcgggcatccccctgtccatgcagaccaaagaccactttgacctgggcatctactccgggctctacgccaccttctactacatgagggagtgctatccctacctgaaggagactcagggctcggtcatcaacttcgcctccggcgccggcctcttcggcaacgtgggtcagtgctcctacgccgccgccaaagagggcatccgcggcctctcccgcgtcgcggccaccgagtggggcaaggacaacatcaacgtcaacgtggtctgccccctggccatgaccgcccagctggagaacttcaagctctcctaccctgaggcctacgagaaaaacctcagaggggtgcccatgggccgcttcggtgaccccgagctggacatcggccgggtctgcgtgcagctcggctcgcccgacttcaagtacatgtccggcgagaccctcaccctggaaggcggcatgggtcagcgcccctag

SEQ ID NO: 22 - protein, 258 aa, Artificial Sequence
  Synthetic polypeptide
MIFDGKVAIITGGGKAKSIGYGIAVAYAKEGANLVLTGRNEQKLLDAKEELERLYGIKVLPLAVDVTPSDESEDRVKEAVQKVIAEFGRIDVLINNAHASASGIPLSMQTKDHFDLGIYSGLYATFYYMRECYPYLKETQGSVINFASGAGLFGNVGQCSYAAAKEGIRGLSRVAATEWGKDNINVNVVCPLAMTAQLENFKLSYPEAYEKNLRGVPMGRFGDPELDIGRVCVQLGSPDFKYMSGETLTLEGGMGQRP